(19) United States
(12) Patent Application Publication
(10) Pub. No.: US 2012/0165221 A1
(43) Pub. Date: Jun. 28, 2012

(54) DIAGNOSIS OF CANCERS THROUGH GLYCOME ANALYSIS

(76) Inventors: Dorit Landstein, Moshav Bitzaron (IL); Allbena Samokovlisky, Ashdod (IL); Boris Gorelik, Mazkeret Batya (IL); Ilana Belzer, Rishon Le Zion (IL); Mor Goldenberg-, Herzlyia (IL); Oshry Biton, Ashdod (IL); Rakefet Rosenfeld, Maccabirn (IL); Shoshy Mizrahy, Tel Aviv (IL); Yeshayahu Yakir, Rishon Le Zion (IL)

(21) Appl. No.: 13/394,167

(22) PCT Filed: Sep. 7, 2010
(86) PCT No.: PCT/IL10/00739

§ 371 Date: Mar. 5, 2012

Related U.S. Application Data
(60) Provisional application No. 61/240,299, filed on Sep. 7, 2009.

(51) Int. Cl. C40B 30/04; G01N 33/574; C08B 37/00

(52) U.S. Cl. 506/9; 536/123.1; 436/501; 435/7.92

(57) ABSTRACT

Markers and methods of diagnosis and monitoring of cancer through global glycome analysis.

### FIELD OF THE INVENTION

The present invention relates to the field of medical diagnostics, and more specifically to biomarkers for diagnosis of cancers, and particularly to biomarkers related to glycome analysis, and kits and methods of use thereof.

### BACKGROUND OF THE INVENTION

Mortality rates of many cancers have not changed dramatically in the last 20 years. Early detection was shown to greatly improve the efficacy of cancer treatment, yet detection is often only possible after the appearance of the first clinical symptoms, which in some cancers occurs too late for successful intervention. This is largely due to the absence of specific and sensitive tests that allow early screening and monitoring of cancerous states. Therefore, the discovery of novel tumor biomarkers is considered to be increasingly critical for improving cancer treatment.

In the past decade, many works have focused on biomarker discovery. One of the most promising sources for biomarker discovery is the human blood, in particular serum and plasma, which can reflect many events in the body, in real time. Yet, despite immense efforts, only a very small number of plasma proteins have been proven to have diagnostic value. Frequently, these biomarkers do not stand alone and are accompanied by other tests for monitoring and diagnosis. Most of these are not specific and sensitive enough for wide screen diagnosis.

Ideally, cancer diagnostic methods should enable the identification of cancer biomarkers in the blood that could be used for one of four purposes: (i) screening a healthy population or a high risk population for the presence of cancer; (ii) developing diagnosis assays of cancer or of a specific type of cancer; (iii) determining the prognosis in a patient; and (iv) monitoring the course in a patient in remission or while receiving surgery, radiation, or chemotherapy.

The current panel of blood biomarkers for cancer consists mostly of specific proteins that are associated with malignancy. No tumor marker now available has met the above ideal tumor marker concept. Certain cancer-associated proteins in blood are detected by specific mAbs (e.g. PSA, CEA, CA-125, CA-19.9). This practice is routinely employed in hospitals, yet has high-frequency of “false positive” failures.

The human genome encodes no more than 30,000-50,000 proteins; this emphasizes the importance of post-translational modifications in modulating the activities and functions of proteins in health and disease (Kim & Varki, 1997). The most widespread and diverse post-translational modification is glycosylation. The unique diverse ability of glycans compared to genome or proteome makes the glycans ideal for diagnosis and monitoring of cancer.

Cancer-associated changes in the glycome of the tumor tissue are very frequent. The location and variation of glycans place them in a position to mediate cellular and intracellular signaling events, as well as participate in different biological processes including pathology states such as cancer (Kim & Varki, 1997). Studies on different types of tumors have shown specific changes in glycosylation with invasion, metastasis, angiogenesis and immunity additional to various stages of the tumor progression (Kobota & Amano, 2005; Dube & BErtozzi, 2005; Peracaula et al., 2003).

Currently glycome-analysis technologies fall behind the rapidly developing genome- and proteome analyzing technologies. Therefore, relatively little progress has been made in the use of differential glycosylation for cancer diagnosis. Therefore, analyses of glycans could be useful as cancer diagnostic and monitoring tools. Identifying glycan-based cancer associated blood markers may lead to development of diagnostic kits for early detection and monitoring of cancer disease via glycan alterations in blood. The current best practice is based on normal phase HPLC followed by exoglycosidase digestion and mass spectrometry analysis. However, these methods are not suitable for clinical laboratory and screening of large amount of serum samples. Thus, glycome-analysis techniques are not currently available in a clinical setting for cancer diagnosis.

### SUMMARY OF THE INVENTION

The background art does not teach or suggest markers for reliable detection and monitoring of cancer, such as gastrointestinal cancers and genitourinary tract cancers, through glycome analysis. The background art also does not teach or suggest glycome based markers for early detection and monitoring of gastrointestinal cancers and genitourinary tract cancers.

The present invention overcomes these drawbacks of the background art by providing markers and methods of diagnosis and monitoring of cancer, preferably for early diagnosis and monitoring, through glycome analysis. According to some embodiments of the present invention, the glycome analysis is performed through lectin based microarrays. The marker is preferably detected in a sample taken from a subject, such as a human patient for example. Optionally and preferably, the lectin-based microarrays are adapted for large scale screening of cancer-associated glycome markers in serum samples, although of course other types of samples may optionally be used as described in greater detail below.

The biomarkers are preferably glycoproteins or any type of glycosylated entity in the sample which react with the below described lectins, for which a list of abbreviations is given below.

Lectin/Antibody Abbreviations and their specificity

| Abbreviation | Lectin/Antibody name | Specificity |
|---|---|---|
| ALAA | Aleuria aurantia lectin | Fucose |
| AOL | Aspergillus oryzae lectin | Fucose |
| Anti-sLeA | Sialyl Lewis A | Sialyl Lewis A |
| CONA | Concanavalin A/Canavalia | High mannose, Bi-antennary |
| | ensiformis (Jackbean) | |
| DC-SIGN | Dendritic Cell-Specific Intercellular | Fucose (Lewis A, Lewis X and Lewis Y) |
| | adhesion molecule-3-Grabbing | |
| | Non-integrin; CD209 | |
| DSA | Datura stramonium (Jimson weed, | Tri/tetra-antennary |
| | thorn apple) | |
| ECL | Erythrina Cristagalli Lectin | N-linked terminal Gal |
| HHA | Hippeeastrum hybrid (Amaryllis) | High mannose |
| HPA | Helix pomatia (Roman or edible | O-linked GalNAc |
| | snail) | |
| LCA | Lens culinaris Agglutinin (Lens | Bi-antennary, Core mannose amplified by |
| | esculenta, lentil) | core fucose (for this lectin, binding to core |
| | | mannose is enhanced by the presence of |
| | | core fucose; when described as a glycan |
| | | or a portion of a glycan herein, it is |
| | | described as “core mannose and core |
| | | fucose” as the motif and/or component of |
| | | the glycan) |
| PHAE | Phaseolus vulgaris (Kidney bean) | Tri-antennary (2-4), Bi-antennary, |
| | | Bisecting |
| PHAL | Phaseolus vulgaris (Kidney bean) | Tri/tetra-antennary, |
| PSA | Pisum sativum Agglutinin (garden | Bi-antennary, Core mannose amplified by |
| | pea) seeds | core fucose (see above) |
| PVL | Psathyrella velutina lectin | N-linked terminal GlcNAc, Sialic acid |
| STL | Solanum tuberosum | High antennarity |
| Siglec-5 | sialic acid binding Ig-like lectin-5 | 2,3 sialic acid |
| Siglec-7 | sialic acid binding Ig-like lectin-7 | Strong on antennary 2,8SA-2,3SA and |
| | | detectable on O-linked 2,3 and 2,6 sialic |
| | | acid |
| UEAI | Ulex europaeus agglutinin I | Fucose |
| WGA | Triticum Vulgaris/aestivum (wheat | Poly-GlcNAc, poly-sialic acid, GalNAc |
| | germ) | |

Reactivity may optionally be to a group of saccharide binding agents, such as a group of lectins for example. A non-limiting list of abbreviations of groups of such lectins, to which reactivity is determined, is given below.

Lectin group composition

| Lectin group | Average signal of: |
|---|---|
| bi2 | CONA LCA PSA |
| bi3 | PHAE LCA PSA |
| bi4 | PHAE LCA PSA PVL |
| core 1 | CONA LCA |
| core11 | same as core 1 |
| core22 | CONA LCA PSA |
| Groups defined in Example 9 | |
| Fucose | UEAI AOL |
| Sialic acid | Siglec-5 Siglec-7 |

In addition, as described below, according to some embodiments of the present invention, the biomarker comprises one or more analytical biomarker functions. These analytical biomarker functions relate to the determination of a ratio or other mathematical relationship between the presence, absence or amount detected of reactivity to a saccharide binding agent, as described in greater detail below.

According to some embodiments, there is provided a biomarker for detecting stomach cancer in a sample taken from a subject, comprising one or more glycans having reactivity to one or more of the following saccharide binding agent combinations: HHA and Anti-sLeA; PSA and bi3; bi2 and bi4; DSA and HPA; STL, ALAA, and Sialic acid group; ECL, ALAA, and DC-SIGN; DSA, ALAA, and DC-SIGN; ALAA, DC-SIGN, and Siglec-5; ALAA, Siglec-5, and Fucose group; or PVL, PSA, and Anti-sLeA; or a combination or a ratio thereof.

Optionally the biomarkers are selected from the following analytical biomarker functions: Model 1-log 2(HHA/Anti-sLeA); Log 2 PSA/Log 2 bi3; log 2(bi2/bi4); log 2(DSA/HPA); and Model 2-log 2(bi2/bi4); log 2(PSA/bi3); log 2(HHA/Anti-sLeA).

Also optionally said glycan comprises a motif selected from the group consisting of Fucose, Sialyl Lewis A; High mannose, Bi-antennary; Tri/tetra-antennary; High mannose; O-linked GalNAc; Core mannose and core fucose; Tri-antennary (2-4), Bi-antennary, Bisecting; Bi-antennary, Core mannose and core fucose; N-linked terminal GlcNAc, Sialic acid; High antennarity; Fucose (Lewis A, Lewis X and Lewis Y); and 2,3 sialic acid. These glycan features correspond to the binding specificities of the saccharide binding agents which were shown, alone or in combination, to be diagnostically discriminatory for gastrointestinal cancer, such as stomach cancer and/or pancreatic cancer, for example. By “saccharide binding agent” it is meant any agent that is capable of specifically binding to a glycan, including but not limited to lectins and antibodies.

By “glycan” it is meant any oligosaccharide, polysaccharide, glycoprotein and the like.

According to other embodiments of the present invention, there is provided use of a combination of saccharide binding agents for detecting stomach cancer in a sample taken from a subject, wherein said combination is selected from the group consisting of HHA and Anti-sLeA; PSA and bi3; bi2 and bi4; DSA and HPA; STL, ALAA, and Sialic acid group; ECL, ALAA, and DC-SIGN; DSA, ALAA, and DC-SIGN; ALAA, DC-SIGN, and Siglec-5; ALAA, Siglec-5, and Fucose group; or PVL, PSA, and Anti-sLeA; or a combination or a ratio thereof.

Optionally the biomarkers are selected from the following analytical biomarker functions: Model 1-log 2(HHA/Anti-sLeA); Log 2 PSA/Log 2 bi3; log 2(bi2/bi4); log 2(DSA/HPA); and Model 2-log 2(bi2/bi4); log 2(PSA/bi3); log 2(HHA/Anti-sLeA).

According to still other embodiments of the present invention, there is provided use of a glycan for detecting gastrointestinal cancer in a sample taken from a subject, the glycan comprising a motif selected from the group consisting of Fucose, Sialyl Lewis A; High mannose, Bi-antennary; Tri/tetra-antennary; High mannose; O-linked GalNAc; Core mannose and core fucose; Tri-antennary (2-4), Bi-antennary, Bisecting; Bi-antennary, Core mannose and core fucose; N-linked terminal GlcNAc, Sialic acid; High antennarity; Fucose (Lewis A, Lewis X and Lewis Y); and 2,3 sialic acid.

Optionally for this use, said glycan is characterized by having reactivity to a saccharide binding agent selected from the group consisting of: ALAA, AOL, Anti-sLeA, CONA, DC-SIGN, DSA, ECL, HHA, HPA, LCA, PHAE, PHAL, PSA, PVL, STL, Siglec-5, Siglec-7, UEAI and WGA.

According to still other embodiments of the present invention, there is provided a kit for detecting gastrointestinal cancer in a sample taken from a subject, comprising a saccharide binding agent having the same saccharide binding specificity as an agent selected from the group consisting of: ALAA, AOL, Anti-sLeA, CONA, DC-SIGN, DSA, ECL, HHA, HPA, LCA, PHAE, PHAL, PSA, PVL, STL, Siglec-5, Siglec-7, UEAI and WGA; and at least one reagent for detecting binding of the saccharide binding agent to the sample taken from the subject.

According to other embodiments of the present invention, there is provided a biomarker for detecting pancreatic cancer in a sample taken from a subject, comprising one or more glycans having reactivity to one or more of the following saccharide binding agent combinations: PSA and core 22; PHAL and core11; WGA and bi3; PHAL and bi2; PSA and bi2; PHAL and core1; PHAE and PHAL; or a combination or a ratio thereof.

Optionally the biomarkers are selected from the following analytical biomarker functions: Model 1-Log 2 PSA/Log 2 core22; Log 2 PHAL/Log 2 core11; Log 2 WGA/Log 2 bi3; log 2(PHAL/bi2). Model 2-Log 2 PSA/Log 2 bi2; Log 2 WGA/Log 2 bi3; Log 2 PHAL/Log 2 core 1; log 2(PHAE/PHAL).

According to other embodiments of the present invention, there is provided a biomarker for detecting pancreatic cancer in a sample taken from a subject, comprising reactivity to a glycan on haptoglobin, wherein said reactivity relates to binding of one or more of HPA, bi1, LCA, WFA, gal-galnac2, Siglec-7.

Optionally the biomarker comprises reactivity to a combination of one or more of HPA and bi1; LCA and HPA; WFA and gal-galnac2; or WFA and Siglec-7.

Optionally the biomarkers are selected from the following analytical biomarker functions: Model 1: log 2(HPA/bi1); log 2(LCA/HPA); log 2(WFA/gal_galnac2); and Model 2: log 2(WFA/gal_galnac2); log 2(WFA/Siglec-7); log 2(LCA/HPA).

According to other embodiments of the present invention, there is provided use of the biomarkers as described herein for diagnosing pancreatic cancer in a sample taken from a subject.

According to other embodiments of the present invention, there is provided a method for diagnosing gastrointestinal cancer in a sample taken from a subject, comprising contacting the sample with a saccharide binding agent as described herein; and if binding is detected, diagnosing the subject with cancer. The method may optionally be used for early diagnosis and/or monitoring.

Optionally, contacting the sample comprises applying the sample to a microarray; and detecting binding of a glycan in the sample to a lectin or antibody on said microarray.

Also optionally said microarray is printed on slides selected from the group consisting of nitrocellulose coated slides, epoxy slides or hydrogel coated slides. By “slide” it is optionally meant any solid support, including but not limited to plates, membranes and the like.

Optionally said gastrointestinal tract cancer comprises stomach cancer or pancreatic cancer (as used herein, the term “gastrointestinal tract” optionally relates to stomach and any other component of the gastrointestinal tract, plus the pancreas).

According to other embodiments of the present invention, there is provided a use, kit or method as described herein, wherein said sample is selected from the group consisting of seminal plasma, blood, serum, urine, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, cyst fluid, broncho alveolar lavage, lavage of the reproductive system and/or lavage of any other part of the body or system in the body, and stool or a tissue sample.

According to other embodiments of the present invention, there is provided a use, kit or method as described herein, wherein said saccharide binding agent is an essentially sequence-specific agent.

Unless otherwise described herein, all biomarkers are present in their isolated form or alternatively are detected in a sample taken from a subject with some type of specific saccharide binding agent which recognizes the biomarker, whether an antibody, lectin or proteins that bind to carbohydrate residues, or any other such binding agent. For example, glycosidases are enzymes that cleave glycosidic bonds within the saccharide chain. Some glycosidases may recognize certain oligosaccharide sequences specifically. Another class of enzymes is glycosyltransferases, which cleave the saccharide chain, but further transfer a sugar unit to one of the newly created ends. For the purpose of this application, the term “lectin” also encompasses saccharide-binding proteins from animal species (e.g. “mammalian lectins”).

A saccharide-binding agent is preferably an essentially sequence-specific agent. As used herein, “essentially sequence-specific agent” means an agent capable of binding to a saccharide. The binding is usually sequence-specific, i.e., the agent will bind a certain sequence of monosaccharide units only. However, this sequence specificity may not be absolute, as the agent may bind other related sequences (such as monosaccharide sequences wherein one or more of the saccharides have been deleted, changed or inserted). The agent may also bind, in addition to a given sequence of monosaccharides, one or more unrelated sequences, or monosaccharides.

The essentially sequence-specific agent is optionally and preferably a protein, such as a lectin, a saccharide-specific antibody or a glycosidase or glycosyltransferase. Examples of saccharide-binding agents lectins include but are not limited to:
lectins isolated from the following plants: Conavalia ensiformis, Anguilla anguilla, Triticum vulgaris, Datura stramoniuim, Galanthus nivalis, Maackia amurensis, Arachis hypogaea, Sambucus nigra, Erythrina cristagalli, Lens culinaris, Pisum sativum, Solanum tuberosum; Glycine max, Phaseolus vulgaris, Allomyrina dichotoma, Dolichos biflorus, Lotus tetragonolobus, Ulex europaeus, Hippeastrum hybrid, and Ricinus communis.
lectins isolated from fungi: Aleuria aurantia; Aspergillus oryzae; Psathyrella velutina
lectins isolated from snail: Helix pomatia (Roman or edible snail)
recombinant human lectins: Dendritic Cell-Specific Intercellular adhesion molecule-3-Grabbing Non-integrin (CD209); sialic acid binding Ig-like lectin-5; sialic acid binding Ig-like lectin-7
antibodies: Anti-Sialyl Lewis A antibody

Other biologically active carbohydrate-binding compounds include cytokines, chemokines and growth factors. These compounds are also considered to be lectins for this patent application. Examples of glycosidases include alpha—Galactosidase, beta—Galactosidase, N-acetylhexosaminidase, alpha—Mannosidase, beta—Mannosidase, alpha—Fucosidase, and the like. Some of these enzymes may, depending upon the source of isolation thereof, have a different specificity. The above enzymes are commercially available, e.g., from Oxford Glycosystems Ltd., Abingdon, OX14 1RG, UK, Sigma Chemical Co., St. Lois, Mo., USA, or Pierce, POB. 117, Rockford, 61105 USA.

The saccharide-binding agent can also optionally be a cleaving agent. A “cleaving agent” is an essentially sequence-specific agent that cleaves the saccharide chain at its recognition sequence. Typical cleaving agents are glycosidases, including exo- and endoglycosidases, and glycosyltransferases. However, chemical reagents capable of cleaving a glycosidic bond may also serve as cleaving agents, as long as they are essentially sequence-specific. The term “cleaving agent” or “cleavage agent” is within the context of this specification synonymous with the term “essentially sequence-specific agent capable of cleaving”.

The cleaving agent may act at a recognition sequence. A “recognition sequence” as used herein is the sequence of monosaccharides recognized by an essentially sequence-specific agent. Recognition sequences usually comprise 2-4 monosaccharide units. An example of a recognition sequence is Gal-beta-1-3 GalNAc, which is recognized by a lectin purified from Arachis hypogaea. Single monosaccharides, when specifically recognized by an essentially sequence-specific agent, may, for the purpose of this disclosure, be defined as recognition sequences.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives”. Subjects who are not diseased and who test negative in the assay are termed “true negatives”. The “specificity” of a diagnostic assay is 1 minus the false positive rate, where the “false positive” rate is defined as the proportion of those without the disease who test positive.

While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above.

In at least some embodiments, the subject invention provides polyclonal and monoclonal antibodies and fragments thereof or an antigen binding fragment thereof comprising a binding site such that the fragment binds specifically to any one of the biomarkers, for example by binding to a specific saccharide motif or glycan as described herein.

The term “antibody” as referred to herein includes whole polyclonal and monoclonal antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen such as a biomarker as described herein. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V Light, V Heavy, Constant light (CL) and CH1 domains; (ii) a F(ab′).2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds a biomarker is substantially free of antibodies that specifically bind antigens other than the biomarker, respectively. An isolated antibody that specifically binds a biomarker may, however, have cross-reactivity to other antigens. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

Optionally and preferably, a combination of antibodies or antigen binding fragments thereof is used to detect a plurality of such specific saccharide motifs or glycans. Optionally, the antibody or antigen binding fragment thereof features a detectable marker, wherein the detectable marker is a radioisotope, a metal chelator, an enzyme, a fluorescent compound, a bioluminescent compound or a chemiluminescent compound.

In at least some embodiments of the present invention, the methods are conducted with a sample isolated from a subject having, predisposed to, or suspected of having the disease, disorder or condition. In at least some embodiments of the present invention, the sample is a cell or tissue or a body fluid sample.

In at least some embodiments, the subject invention therefore also relates to diagnostic methods and or assays for diagnosing a disease optionally and preferably in a biological sample taken from a subject (patient), which is more preferably some type of body fluid or secretion including but not limited to seminal plasma, blood, serum, urine, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, cyst fluid, broncho alveolar lavage, lavage of the reproductive system and/or lavage of any other part of the body or system in the body, and stool or a tissue sample. The term may also optionally encompass samples of in vivo cell culture constituents. The sample can optionally be diluted with a suitable eluant before contacting the sample to an antibody and/or performing any other diagnostic assay.

According to at least some embodiments of the present invention there are provided diagnostic methods that include the use of any of the foregoing saccharide binding agents according to at least some embodiments of the present invention, by way of example in immunohistochemical assay, radioimaging assays, in-vivo imaging, positron emission tomography (PET), single photon emission computer tomography (SPECT), magnetic resonance imaging (MRI), Ultra Sound, Optical Imaging, Computer Tomography, radioimmunoassay (RIA), ELISA, slot blot, competitive binding assays, fluorimetric imaging assays, Western blot, FACS, bead, and the like.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

As used herein, the indefinite articles “a” and “an” mean “at least one” or “one or more” unless the context clearly dictates otherwise.

Unless otherwise defined, all technical and scientific terms used herein have the same meaning as commonly understood by one of ordinary skill in the art to which this invention pertains. In case of conflict, the patent specification, including definitions, will control.

### BRIEF DESCRIPTION OF THE FIGURES

Some embodiments of the invention are herein described, by way of example only, with reference to the accompanying figures. The description, together with the figures, makes apparent how embodiments of the invention may be practiced to those skilled in the art. It is stressed that the particulars shown in the figures are by way of example and for purposes of illustrative discussion of embodiments of the invention.

In the figures:

FIG. 1 shows the results from fingerprints of pooled human serum that were treated enzymatically and analyzed on the lectin array;

FIG. 2 demonstrates fingerprints of various serum samples;

FIG. 3 shows age distribution of the patients in the different study subsets during the global glycosylation analysis.

FIG. 4 shows age distribution of pancreatic cancer patients in the different study subsets during the global glycosylation analysis.

FIG. 5 shows predicted probability of a sample to belong to the stomach cancer group as function of the actual level of validation set patients;

FIG. 6 shows the predicted probability of a sample to belong to the pancreas cancer group as function of the actual level of validation set patients;

FIG. 7 shows age distribution of the patients in the different study subsets during haptoglobin glycosylation analysis; note that due to the different randomization, this distribution differs from the one depicted on FIG. 4;

FIG. 8 shows the predicted probability of a sample to belong to the pancreas cancer group as function of the actual level of validation set patients;

FIG. 9 shows immunoprecipitated PSA from prostate cancer patient serum;

FIG. 10 shows the flow of test samples over the study for stomach cancer in a schematic diagram;

FIG. 11 shows the experimental flow for the study for stomach cancer; and

FIG. 12 shows updated validated results for stomach cancer for some non-limiting groups of lectins.

### DESCRIPTION OF EMBODIMENTS

The present invention provides, in at least some embodiments, markers and methods of diagnosis and monitoring of cancer, preferably for early diagnosis and monitoring, through glycome analysis. According to some embodiments of the present invention, the glycome analysis is performed through lectin based microarrays. The marker is preferably detected in a sample taken from a subject, such as a human patient for example, for example by detecting reactivity to a lectin or combination of lectins. Optionally and preferably, the lectin-based microarrays are adapted for large scale screening of cancer-associated glycome markers in serum samples, although of course other types of samples may optionally be used as described in greater detail below. The lectin array can be enhanced with antibodies directed against glycan structures, such as the Lewis epitope.

As described herein, non-limiting examples of such biomarkers include those which are useful for diagnosis of gastrointestinal cancer, such as stomach cancer or pancreatic cancer for example.

Pancreatic adenocarcinoma represents the imperative role of early diagnostics of cancer. Pancreatic adenocarcinoma is the fifth leading cause of cancer death and has the lowest survival rate for any solid cancer (Goggins, 2005; DiMagno et al, 1999; Jemal et al, 2003). Patients with surgically excised pancreatic cancers have the best hope for cure as they can achieve a 5-year survival of 15-40% after pancreaticoduodenectomy (Yeo et al, 1995). Unfortunately, only 10-15% of firstly diagnosed patients present with small, excisable cancers (DiMagno et al, 1999). Therefore, early diagnostics of pancreatic cancer in routinely taken blood samples could increase the proportion of patients diagnosed with pancreatic cancer being in a respectable stage and thus significantly increase their 5-years survival from 3-4% to 15-40%.

Stomach (gastric) cancer, is the fourth most common cancer and the second most cause of cancer-related death world-wide. Gastric cancer accounts for nearly 1,000,000 new cases and over 850,000 deaths annually (Pisani et al. 1999). Gastric cancer is often asymptomatic or causes only non-specific symptoms in its early stage. By the time when more severe symptoms occur the prognosis is poor. Currently, there is no specific and sensitive biomarker for early detection. An invasive method, endoscopic evaluation, is the golden standard for diagnosis of gastro-intensinal diagnosis neoplasm (Lam & Lo, 2008).

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### Examples

### Example 1

### Gastrointestinal Cancer I

This Example relates to global glycome analysis for the detection of gastrointestinal cancer, particularly stomach cancer and pancreatic cancer. Rather than concentrating on a single biomarker, this Example demonstrates the overall global analysis of the glycome in order to discover one or more specific glycome features which may then be used for cancer diagnosis. For this non-limiting example, nitrocellulose coated slides were used. For other examples below, different materials were used as indicated.

### Methods

### Serum Samples

The serum samples used were of Caucasian patients that are “drug naïve”, for which comprehensive demographic and clinical data were available. The samples were supplied by two different sources: RNTECH (France) and Asterand (USA).

### Sample Preparation

Serum samples were depleted of 14 most abundant proteins using IgY-14 spin column (GenWay, San Diego), according to manufacturer instructions. At all stages 1×PBS was used instead of the Tris-HCL buffer recommended by the manufacturer. Basically, 15 ul of serum was diluted in 1×PBS to final volume of 500 ul. The diluted serum was filtered by Spin-X (Costar) and the strained serum was loaded onto the depletion IgY-14 column. The unbound fraction (depleted serum) was collected. The bound proteins were eluted and neutralized using the kit stripping and neutralization buffers. The column was regenerated for further use (up to 100 times).

The depleted serum was fluorescently labeled (final Fluorophor/Protein=1) using Cy3—NHS (Amersham). Following incubation for 2 hours at 4° C. on an end-over-end shaker the reaction was stopped with 100 ul of 1M Tris-HCl pH 7.5 per 1 ml. NAP-5 columns (Amersham), equilibrated with 10 ml with PBS, were used to separate free Cy3 from labeled sample.

### Glycoanalysis

The lectin arrays used were comprised of nitrocellulose coated glass slides (GraceBio) printed with various lectins from different sources such as plant, human, etc, as well as antibodies to glycan epitopes. Slides were processed in 6 chambers trays. The minimal experiment requires one CS and one sample slide. The required solutions and volumes for the amount of slides processed in the experiment were prepared. Cy3-labeled depleted serum sample was prepared in wash buffer containing 1×PBS, 0.4 mM MgCl₂, 0.4 mM CaCl₂, 0.004M MnCl₂, 0.0009% Triton X-100 to a final protein concentration of 15 ug/ml.

### Procedure

An Incubation frame was adhered onto each lectin array that was processed, flush with edges of the slide. Lectin arrays were handled carefully, wearing non-powdered gloves during slide handling and avoiding any contact with the membrane-covered surface.

The slide(s) were placed membrane side up in a 6 chambers tray. Pre-wetting solution containing 1×PBS, 0.4 mM MgCl2, 0.4 mM CaCl2, 0.004M MnCl2 (20 ml) was added and slides were incubated on an orbital shaker for 5 minutes. Pre-wetting solution was removed and 20 ml complete blocking solution containing 1×PBS, 0.4 mM MgCl2, 0.4 mM CaCl2, 0.004M MnCl2, 1% BSA, 0.0009% Triton X-100 was added to each chamber, which was then incubated on an orbital shaker set to rotate at 50 rpm for 60 min at room temperature (15-25° C.). Blocking solution was discarded.

Arrays were washed by adding 20 ml complete wash solution to the chamber, incubating on an orbital shaker set to rotate at 50 rpm for 5 min at room temperature (15-25° C.), and discarding wash solution. The wash step was repeated twice more. After the third wash step, the arrays were left submerged in wash solution to prevent them drying out.

A single array was then taken from the chamber and wash solution removed by pressing a paper towel to the back and edges of the array, taking care not to touch the membrane. The array was placed in a clean chamber and a 450 μl sample was pipetted onto the membrane, ensuring that the membrane is fully covered, without touching the membrane, and avoiding formation of bubbles on the membrane. The procedure was repeated for the remaining arrays.

Arrays were incubated in the dark on an orbital shaker set to rotate at 50 rpm for 60 min at room temperature (15-25° C.). The trays were kept covered at all times to minimize evaporation and light in order to prevent drying out of slides and bleaching of fluorescence.

Arrays were washed in the dark by adding 25 ml complete wash solution to the chamber, placing on an orbital shaker set to rotate at 50 rpm for 5 min at room temperature (15-25° C.), and discarding wash solution. The wash procedure was repeated twice more. After the third wash step, the incubation frame was carefully peeled from each array. The arrays were washed in the dark for 1 min with 25 ml RO- or HPLC-grade water, and dried. The arrays were scanned and analyzed.

Drying Slides after Processing

To avoid nonspecific background signals, slides were dried before scanning.

Slide(s) were removed from final water wash, and the back of the slide(s) wiped gently with a laboratory wipe. The slides were centrifuged at 200×g for 5-10 min (or until slides are dry) in a Coplin jar or a centrifuge slide carrier, then air dried in the dark until membrane is completely white.

### Scanning Slides

Following sample processing and drying, slides were scanned using a microarray Laser scanner with adjustable laser power and photomultiplier tube (PMT), (Axon GenePix 4200) with Cy3 filter. Images were analyzed using image analysis software: Array-Pro™ ver. 4.5 (Media Cybernetics, Inc) and PPIP ver 2.0 (Procognia Proprietary Image Processing, Procognia, Ltd).

Results

In this project, the use of microarrays for glycoanalysis of purified glycoproteins was expanded to the analysis of complex protein mixtures from serum. Untreated serum contains about 30-50 mg/ml of albumin and IgG. To enable detection of lower abundance serum proteins a method for removal of 14 most abundant serum/plasma proteins by a mixed antibodies commercial spin column (Seppro IgY-14, GenWay) was established. The antibodies in the column are directed against HSA, IgG, Fibrinogen, Transferrin, IgA, IgM, Apo A-I, Apo A-II, Haptoglobin, alpha-1-Antitripsin, alpha-1-Acid Glycoprotein, alpha-2-Macroglobulin, complement C3 and LDL. In addition a method for Cy3 labeling of serum proteins and dye clean-up was calibrated. Finally, optimal protein concentration for analysis on the lectin microarray was determined and the entire processing protocol was established.

The lectin array consists of a set of 20-30 lectins printed on a membrane-coated glass slide in a range of concentrations that provide a dose-response for each printed lectin. When sample of intact purified glycoprotein is applied to the array, and its binding pattern is detected by direct labeling using fluorophore, the resulting fingerprints are highly characteristic of the glycosylation pattern of the sample. The large number of lectins, each with its specific recognition pattern, ensures high sensitivity of the fingerprint to changes in the glycosylation pattern. The lectins on the array are grouped according to their monosaccharide specificities, in cases where possible; lectins in the group that is denoted “complex” do not bind monosaccharides, but bind complex N-linked glycans. The groups and differences between lectins within each group are detailed below.

Complex

The lectins in this group recognize branching at either of the two α-mannose residues of the tri-mannosyl core of complex N-linked complex glycans. Some of the lectins of this group are sensitive to different antennae termini as they bind large parts of the glycan structure. The lectins denoted Complex(1) and Complex(4) have a preference for 2,6-branched structures; lectin Complex(3) has a preference for 2,4-branched structures, and lectin Complex(2) recognizes with similar affinity both structures.

GIcNAc

The lectins in this group bind N-acetylglucosamine (GIcNAc) and its β4-linked oligomers with an affinity that increases with chain length of the latter. The carbohydrate-specificity of both lectins in this group do not differ, yet differences in their binding patterns are observed and probably stem from the non-carbohydrate portion of the samples.

Glc/Man

This group of lectins is a subgroup of the mannose binding lectins (see below), and are denoted Glc/Man binding lectins since they bind, in addition to mannose, also glucose. All of the lectins in this group bind to bi-antennary complex N-lined glycans with high affinity. In comparison to their affinity for bi-antennary structures, lectins Glc\Man(1) and (2) bind high mannose glycans with lower affinity, whereas lectin Glc\Man(3) will bind high mannose glycans with higher affinity.

Mannose

This group consists of lectins that bind specifically to mannose. These lectins will bind high mannose structures and, with lower affinity, will recognize the core mannose of bi-antennary complex structures.

Terminal GlcNAc

This lectin specifically recognizes terminal GIcNAc residues.

Alpha Gal

These lectins bind terminal α-galactose (a-Gal). Lectin Alpha-Gal(1) binds both α-galactose and α-GalNAc (α-N-acetylgalactosamine) and may bind to both N and O-linked glycans. Lectin Alpha-Gal(3) binds mainly the Galili antigen (Gala1-3Gal) found on N-linked antennae.

Beta Gal

These lectins specifically bind terminal (non-sialylated) β-galactose residues.

Gal/GaINAc

These lectins are specific for terminal galactose and N-acetyl-galactoseamine residues.

The different lectins within this group differ in their relative affinities for galactose and

N-acetyl-galactoseamine. Lectins (2) and (5) from this group bind almost exclusively Gal; lectins (1), (3) and (4) bind almost exclusively GaINAc. The relative affinities for GaINAc/Gal for the remaining lectins in the group are ranked: (8)>(7)>(6).

Fucose Lectins from this group bind fucose residues in various linkages.

Lectin Fucose(6) binds preferentially to 1-2-linked fucose; Lectin Fucose(8) binds preferentially to 1-3 and 1-6 linked fucose; Lectins Fucose(12) and (13) bind preferentially to Fuel-4GIcNAc (Lewis A antigens).

These lectins generally do not bind the core fucose of N-linked oligosaccharides on intact glycoproteins due to steric hindrance.

Sialic Acid

The sialic acid lectins react with charged sialic acid residues. A secondary specificity for other acidic groups (such as sulfation) may also be observed for members of this group. Lectin Sialic Acid(1) recognized mainly 2-3-linked sialic acid; Lectin Sialic Acid(4) recognizes mainly 2-6-linked sialic acid.

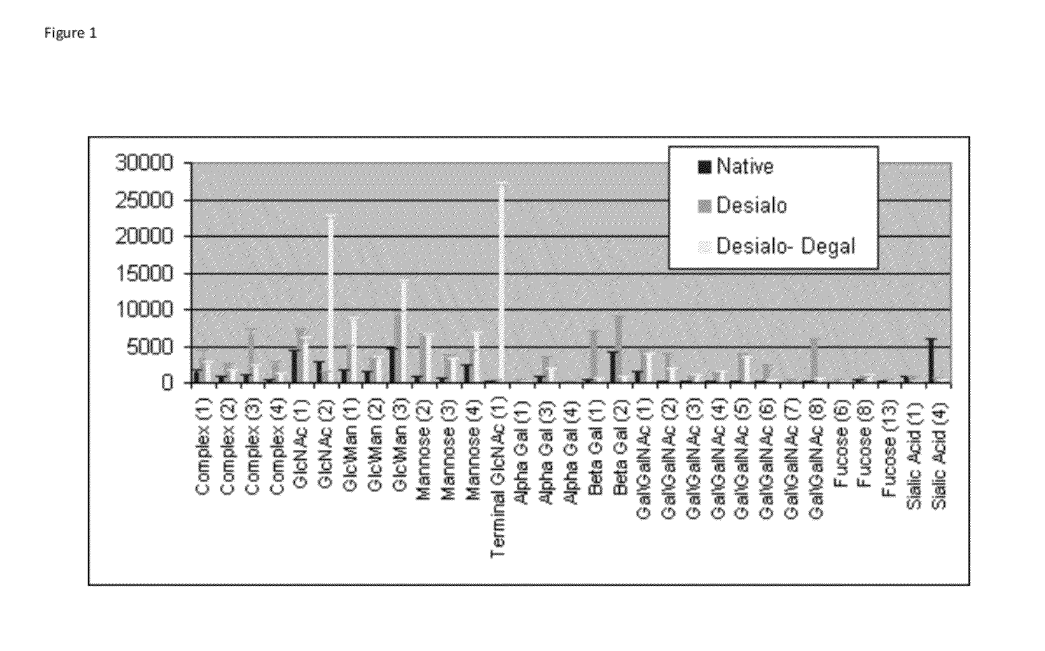

Analyses of fingerprints from Cy3-labeled depleted serum, treated enzymatically for modification of glycans provided biochemical proof of concept for global glycoanalysis of protein mixtures. This is based on our knowledge of lectin specificities that enables us to predict the changes in fingerprints following these modifications. For example, treatment of human serum with Neuraminidase led to reduced signals from Sialic acid lectins and increased signals from terminal beta-gal lectins. Further enzymatic removal of galactose resulted in decreased signals from terminal gal binding lectins and increased signals from GlcNac recognizing lectins, as shown in FIG. 1, which shows the results from fingerprints of pooled human serum that were treated enzymatically and analyzed on the lectin array. Each bar on the X-axis represents binding of the sample to a specific lectin; lectins are coded and grouped according to their specificities. Results of the lectin array binding data for the enzymatically treated serum demonstrate that the lectin microarray technology can be applied to complex mixtures of proteins.

In order to enable detection of cancer-related glycan epitopes which are not recognized by the above described standard arrays, various antibodies and mammalian lectins (anti Lewis antibodies, Siglecs and Selectins) were printed on the arrays with the standard set of lectins. For the new antibodies and mammalian lectins we tested various printing conditions in order to optimize their activity on the array. Support for the specificity of the new binding agents on the array was obtained by comparing native to desialylated samples. The results of analyzing the serum samples on the enhanced arrays demonstrated that the new binding agents were specific.

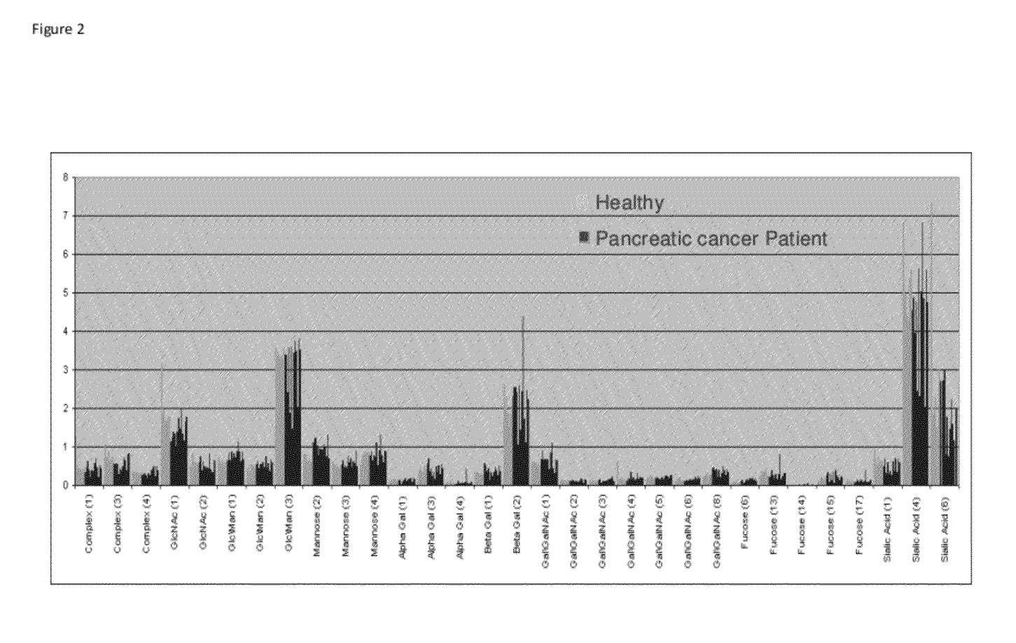

Evaluation of global glycosylation differences between healthy and cancer patient serum was performed with sera from control, and stomach and pancreatic cancer patients. The cancer samples were taken from different stages of disease. All sera samples tested were depleted of 14 most abundant proteins. A comparison of representative fingerprints obtained with pancreatic cancer and control sera is shown in FIG. 2, which demonstrates fingerprints of various serum samples. Results of 7 healthy and 13 pancreatic cancer patient sera are shown.

Lectin microarray binding data of depleted sera were collected. In order to eliminate lot to lot variation between various batches of printed slides a calibration standard (CS) sample was used in each assay. This CS consists of pooled commercial human serum (Sigma), prepared as all the tested sera and pooled to large quantity. Signals from samples were corrected according to the signals from the CS sample in the assay. The parameters used to construct the classification were based on lectin signals obtained from the microarray. The variables used to construct the classification were all ratios between all pairs of lectin signals and lectin group averages, groups being defined by their specificities, and various functions of these ratios. The entire data set was subjected to bioinformatics analysis (see Bioinformatics Example below).

Discussion

Early and accurate detection of GI cancers offer the best hope of cure for the diseases. Glycosylation alterations on specific serum proteins associated with various cancer types and states have been reported (Peracaula et al, 2003a; Hamid et al., 2008; Peracaula et al, 2003). The approach described in this work is unique since it examines glycosylation alterations on mixture of medium and low abundant proteins in serum. Changes in glycosyl transferase and other sugar modifying enzymes have been shown in cancerous states (Arnold et al., 2008). It is therefore reasonable to assume that glycosylation pattern alterations may be found on many serum proteins and not limited to few biomarkers. The high accuracy found in separation between control and cancer patients suggest that global glycosylation analysis on lectin array can be developed and used for cancer diagnosis, monitoring and prognosis.

The development of the technology for global glycoanalysis of protein mixtures can lead to development of a kit for analysis of serum with special relevance for cancer for use in clinical, academic and industrial platforms. Such a kit enables the high-throughput glycoanalysis of glycoproteins on lectin microarrays. This technology is more rapid, as it is performed on the whole glycoprotein, easier to handle, requires only low sample amounts and is cheaper than the traditional analysis methods.

### Example 2

### Bioinformatics Glycome Analysis for Gastrointestinal and Pancreas Cancer

This Example relates to the bioinformatics approach used for global glycome analysis in Example 1, again using nitrocellulose slides. It should be noted that the biomarkers and methods of use thereof as described in Example 1 are not limited by the particular bioinformatics approach but instead may be used independently of this approach. Similarly, this bioinformatics approach may optionally be used for elucidating any type of cancer biomarker through global analysis of the glycome.

A method for the analysis of serum samples using the above described lectin microarray was established. Evaluation of global glycosylation differences in serum samples of healthy versus pancreatic and stomach patient was performed. The samples were taken from patients at different stages of stomach and pancreatic cancer.

Classification of blood samples using patterns of plasma proteins is a multifactor problem. Solving such a problem requires extensive data mining efforts and is prone to overfitting of the models to the data. This problem was addressed according to various non-limiting, illustrative methods as described herein, including cross-validation, blind tests, adding noise to the input data and using multiple data mining methods during the training process.

Computational Methods

Input Data

Serum samples from stomach and pancreas, as well as from control patients were obtained from two suppliers: RNTech and Asterand. Patients are considered as control if they have neither cancer nor other target organ (stomach and pancreas) disease. In addition to these controls, several serum samples from patients with benign stomach (ulcer) or pancreas (pancreatitis) diseases were also obtained from RNTech.

Patients' demographic (age, gender, etc) and clinical data (only partly used at this stage) are also available. All patient sera were collected prior to medical treatment.

Signals obtained from each lectin following fluorescently labeled serum sample binding to the lectin array were collected. The available data are described separately.

Data Preparation

Each experiment produced lectin signal profiles of 12-16 samples. One out of these profiles originated from commercially pooled normal human serum purchased from Sigma. This sample served as reference point that enables accounting for inter-experiment; inter slide lots and other variation factors (Calibration standard, CS, samples). Scanning quality of each profile was assessed using a set of objective measurements. To create the reference point for all slides, glycoprofiles of CS sample obtained from single-batch of slides were obtained. The common reference point (referred as “gold standard”, GS) was calculated by averaging the values of the respective lectin signals. Ratios between the lectin signals in any current CS to those in GS served to correct the corresponding lectin signals in these experiments. In addition all the available lectin signals were normalized to the total signal. Whenever the term “signal” is used, the term refers to the normalized values of the signal.

The available data was expanded in the following ways: signals of lectins that demonstrate specificity to various glycan groups were averaged (e.g. Core0, Core1, Bi1 etc), as specified in the Appendix; log₂ of lectin signals was calculated; log₂ of ratio between any two lectins. Non-finite numbers that may have been produced by the expansion process (e.g. by division by zero) were marked as not a number (NaN). For simplicity, all the resulting columns that contained NaN values were removed.

Parameter Selection Process

General Design

Profiles of the patients younger than 40 years were discarded from the analysis. This threshold is not expected to decrease the validity of the presented analysis and conclusions due to the much higher median diagnosis ages (as described earlier).

The remaining patients were divided into two overlapping groups: (1) gastric cancer and control patients and (2) pancreas cancer and control patients. Samples of benign patients were not included in any of the groups. The two subsets share the same control patient samples. Each group was then randomly divided into training (˜70%) and validation (˜30%) sets. The data expansion process results in a huge hyperspace of more than 2000 parameters (or predictors). Scanning all the possible combinations of these predictors for the best available separation is practically infeasible. Thus, a parameter selection algorithm is required. Principal component analysis (PCA) and similar techniques are widely used for parameter reduction. It is possible to quantify the degree of association of a certain independent attribute (predictor) to the predicted value. We used information gain and Gini gain scores. Each attribute was scored using both methods, followed by averaging the ranks obtained by these methods (consensus scoring). We then select the first 100 best ranking parameters. The number 100 was chosen arbitrarily to enable a reasonable trade-off between parameter diversity and our ability to complete the subsequent steps in a reasonable amount of time. The next step is to select several out of the 100 predictors such that the performance of the resulting model is maximized. Model performance was assessed as follows: any selected attribute set is used to generate Bayesian and decision tree binary classifiers using six-fold cross-validation of the training set. The average values of Matthews Correlation Coefficient (MCC) [14] serves and a quantitative measurement of model performance. We used average of two classifiers, instead of picking a single one in order to minimize the possibility of over-fitting. MCC maximization process was performed using Genetic Algorithm (GA) [15,16]. MCC is a measure of quality of binary prediction and is defined as follows:

$$MCC = \frac{TP \times TN - FP \times FN}{\sqrt{( TP + FP ) ( TP + FN ) ( TN + FP ) ( TN + FN )}}$$

Where TP and FP are the number of true and false positive predictions, respectively; and TN and FN are the number of true and false negative predictions. In terms of our study, control cases are considered as negative, while cancer cases—as positive predictions.

Similarly to Pearson correlation, MCC values range from 1.0 (ideal prediction), through 0 (random prediction) to −1.0 (reversed prediction).

In order to further minimize the over-fitting we have limited the GA to pick not more than four predictors. Chromosome encoding, mutation and cross-over operators, as well as the GA parameters are described in detail in the Appendix.

Due to the stochastic nature of GA, the optimization process was performed for 200 times, resulting in a population of 200 models. The appearance of each model attribute in this population is counted and each model is scored according to the average attribute prevalence.

In the next step we sort the 200 models according to the GA score. If two models had identical value of GA score, the one with higher average attribute prevalence is scored higher. We then take two best scoring models and test them on our validation set samples.

Results Assessment

Our main measure for predictive power assessment is MCC. We also report sensitivity (Sens), specificity (Spec) and positive prediction value (PPV). Those are calculated as follows (using the same abbreviations as in formula for MCC):

$$Sens = 1 - \frac{FN}{TP + FN}$$

$$Spec = 1 - \frac{FP}{FP + TN}$$

$$PPV = \frac{TP}{TP + FP}$$

Results Validation

We have randomly re-assigned the recorded classification of serum samples, keeping the total number of control and gastric/pancreatic cancer patients. We expect a sharp decline in the predictive power of models built and tested with such a data.

In addition, we have repeated the validation procedure after adding a random uniform to the lectin signal data. We tested two noise levels: 10% and 90% of the respective original signal intensity. The predictive power of a well-defined model is not expected to decrease significantly with the smaller noise level. However, the latter case should produce nearly random results.

Results

Demographic Analysis

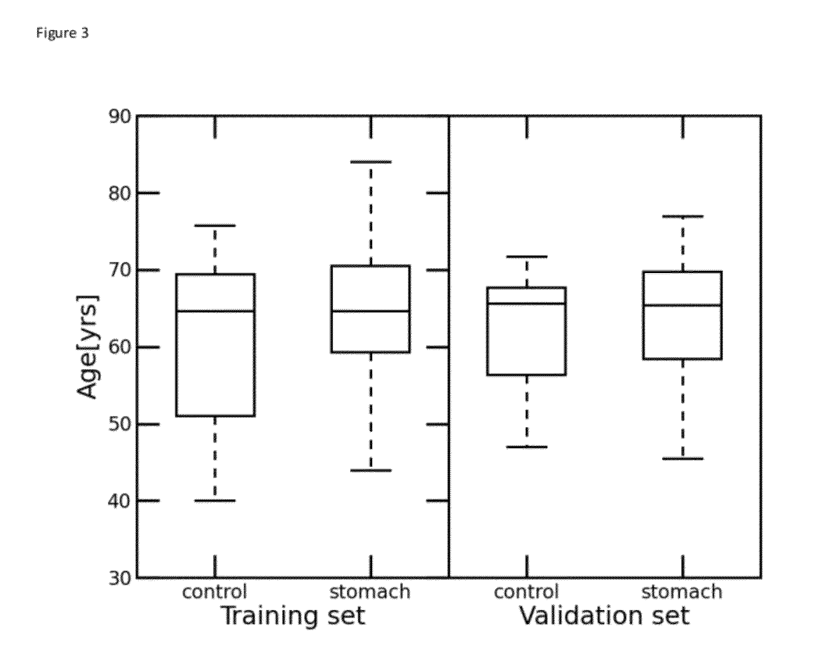

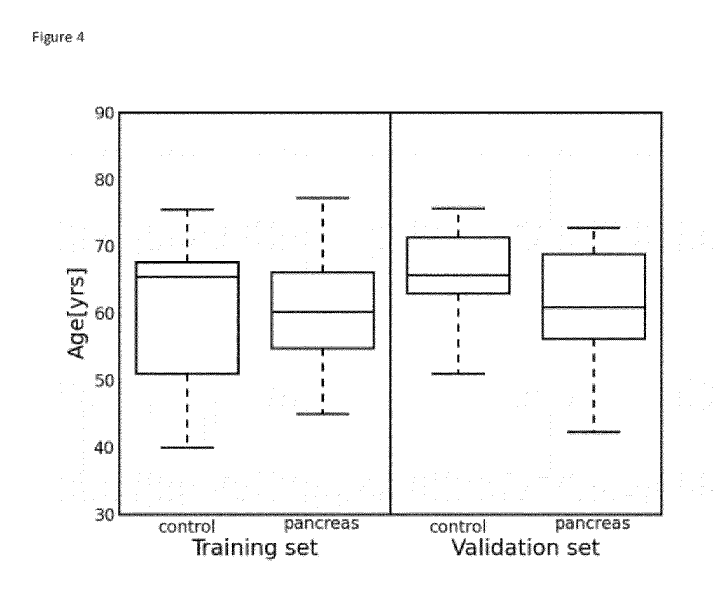

Demographic characteristics of the study population are summarized in Table 1. Age distribution is shown in FIGS. 3 and 4. FIG. 3 shows age distribution of the patients in the different study subsets. Box boundaries correspond to the lower and upper quartile values. Horizontal line inside the boxes represents the median. The whiskers show the range of the data. FIG. 4 shows age distribution of pancreatic cancer patients in the different study subsets. Boxplot conventions are similar to those in FIG. 3.

TABLE 1
Demographic characteristics of the study population. Age is
shown as mean (+/−stdev). Several plasma samples
were glycoprofiled more than once. Number of patients, as
well as the number of glycoprofiles are reported in the table

| | |
|---|---|
| Training | Validation |

| | | | |
|---|---|---|---|
| Cancer | Control | Cancer | Control |

Gastric cancer

| | | | | |
|---|---|---|---|---|
| Age | 64.5 | 60.7 | 63.1 | 62.5 |
| | (7.7) | (10.7) | (9.1) | (7.7) |
| Patients | 66 | 25 | 33 | 7 |
| out of them | | | | |
| male | 39 | 12 | 24 | 4 |
| female | 27 | 13 | 9 | 3 |
| Glycoprofiles | 91 | 50 | | |
| out of them | | | | |
| male | 57 | 25 | 35 | 7 |
| female | 34 | 25 | 12 | 5 |

Pancreatic cancer

| | | | | |
|---|---|---|---|---|
| Age | 60.8 | 59.4 | 61.3 | 64.8 |
| | (7.5) | (10.6) | (8.7) | (8.1) |
| Patients | 37 | 22 | 17 | 10 |
| out of them | | | | |
| male | 18 | 12 | 9 | 4 |
| female | 19 | 10 | 8 | 6 |
| Glycoprofiles | 39 | 39 | 19 | 23 |
| out of them | | | | |
| male | 20 | 22 | 9 | 10 |
| female | 19 | 17 | 10 | 13 |

### Distribution of Calibration Signals

In order to be able to compare glycosylation profiles obtained with different plates and slides, we have analyzed the distribution of CS signals over all the available samples. This analysis (data not shown) indicates that, run number 11552 resulted in a substantial number of lectin outliers. This run was performed with plate k-12-05-08, which was selected for Gold standard creation. Thus, the run 11552 was completely removed from the Gold standard creation. Nevertheless, this run was included in the subsequent analysis under the assumption that the outlying signals would be corrected by the standard correction procedure.

### Distribution of Normalized and Corrected Signals

Having normalized and corrected the signals using Gold standard, we expect glycoprofile of a sample performed at different conditions to be similar. Generally the signals of all the lectins, except to SNA and CONA were reasonably stable.

### Data Mining Results

### Selecting Attributes

The two best scoring models for stomach cancer consist of the following attributes: Model 1: log 2(HHA/Anti-sLeA); Log 2 PSA/Log 2 bi3; log 2(bi2/bi4); log 2(DSA/HPA). Model 2: log 2(bi2/bi4); log 2(PSA/bi3); log 2(HHA/Anti-sLeA).

The two best scoring models for pancreatic cancer consist of the following attributes: Model 1: Log 2 PSA/Log 2 core22; Log 2 PHAL/Log 2 core11; Log 2 WGA/Log 2 bi3; log 2(PHAL/bi2). Model 2: Log 2 PSA/Log 2 bi2; Log 2 WGA/Log 2 bi3; Log 2 PHAL/Log 2 core 1; log 2(PHAE/PHAL).

### Predictive Power

Despite the fact that two methods were used during the training process (Bayes classifier and decision trees), the validation results are reported in term of Bayes classifier only, as it consistently produced the best performance. The performance of the models built with the attributes listed above is detailed in Table 2 (gastric cancer) and Table 3 (pancreatic cancer).

TABLE 2
Performance results for gastric cancer model.

| Noise | | | | |
|---|---|---|---|---|
| (%) | MCC | Sens | Spec | PPV |
| 0 | 0.85 | 0.96 | 0.92 | 0.98 |
| 10 | 0.39 | 0.96 | 0.33 | 0.85 |
| 90 | None | 1 | 0 | 0.8 |

Abbreviations are:
CA—accuracy,
MCC—Matthews correlation coefficient,
Sens—sensitivity,
Spec—specificity,
AUC—area under receiver operating characteristic curve;
PPV—positive predictive value.
Note that due to the random nature of the noise, the results for the “noisy” models may vary.
MCC calculations that result in division by zero are marked as “None”.

TABLE 3
Performance results for pancreas cancer model.

| Noise | | | | |
|---|---|---|---|---|
| (%) | MCC | Sens | Spec | PPV |
| 0 | 0.76 | 0.79 | 0.96 | 0.94 |
| 10 | 0.43 | 0.95 | 0.43 | 0.98 |
| 90 | None | 1 | 0 | 0.45 |

Abbreviations are similar to those in Table 2.

As one may see from these results, the presented models show good to excellent predictive properties. The gastric cancer models are generally more specific and sensitive, compared to those for pancreatic cancer. One possible explanation for this phenomenon is the fact that more plasma samples were available for this indication. Detailed model predictions are listed in Table 4 and Table 5 in the appendix.

### Robustness Tests

Re-shuffling the patients' classifications as described in the “Computational methods” section resulted in failure of the training process to generate a model with a reasonable predictive power. The absolute MCC values of the gastric and pancreatic cancer models in the validation set samples did not exceed 0.2, indicating random or near-random classification (data not shown).

As expected, adding a random noise to the validation set data resulted in decreased performance, as measured by MCC. In the case of pancreas cancer model MCC value of the predictive model decreased from 0.72 to 0.43 after adding 10% noise. Due to division by zero, MCC could not be calculated in the 90% noise case. In the gastric case model 10% noise resulted in MCC value of 0.39, compared to 0.85 without the noise. As in the pancreas cancer case, MCC could not be calculated in the 90% noise case. Note that due to the random nature of the noise, the results for the “noisy” models may vary

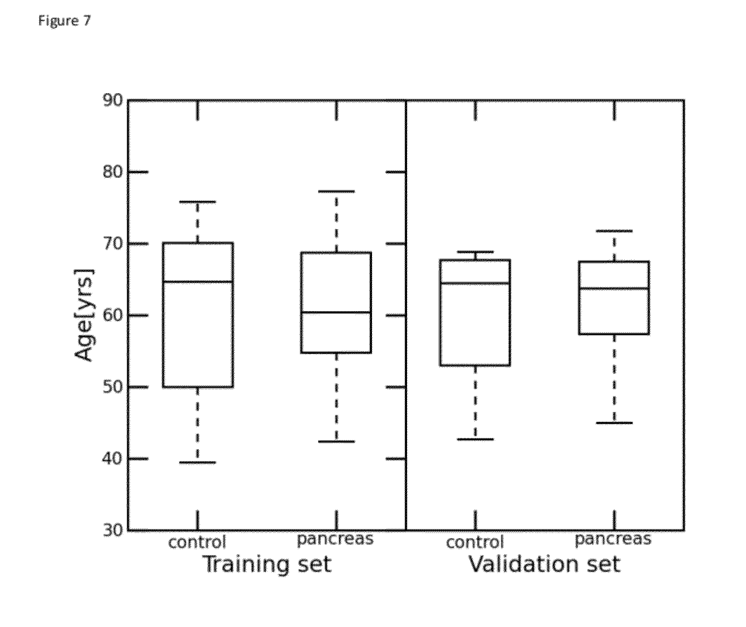

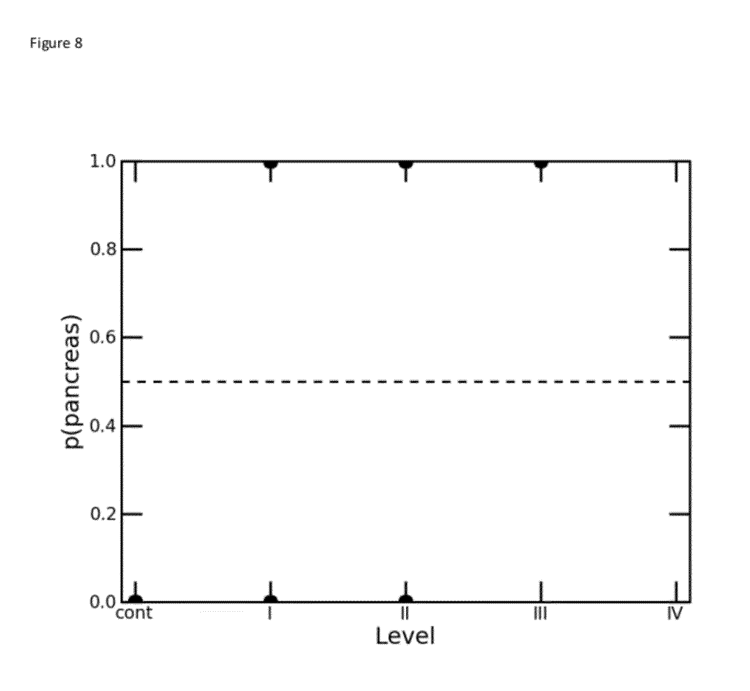

Model predictions do not depend on the patient cancer stage, as is demonstrated on FIGS. 7 and 8. This finding suggests that these models are suitable for early cancer detection.

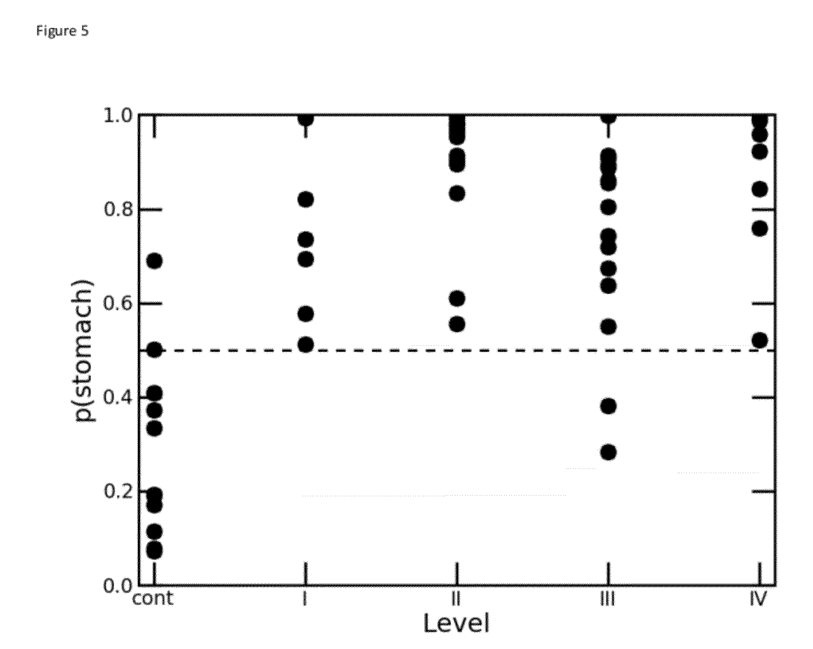

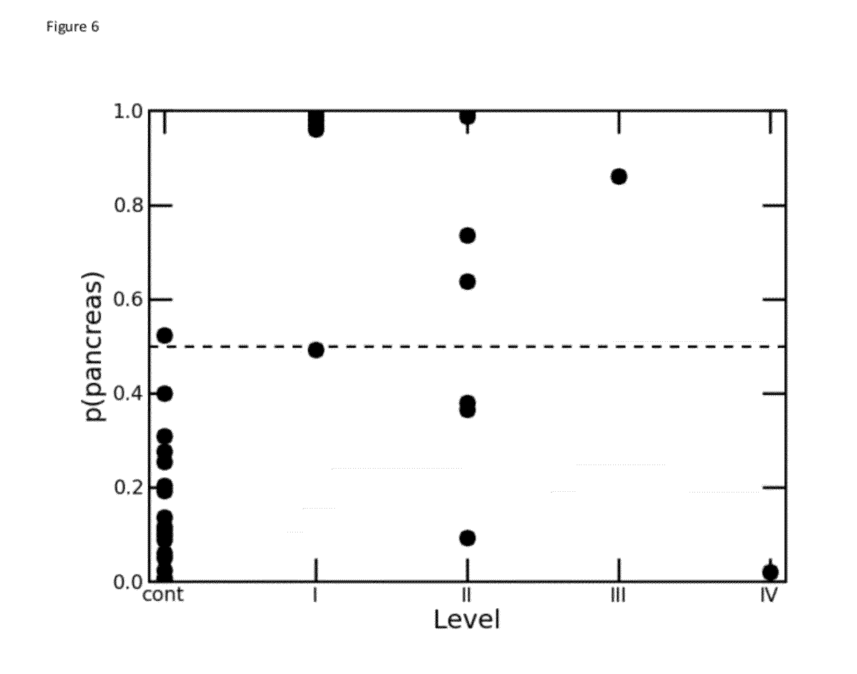

FIG. 5 shows predicted probability of a sample to belong to the stomach cancer group as function of the actual level of validation set patients. Prediction threshold (p=0.5) is marked as dashed line. FIG. 6 shows the predicted probability of a sample to belong to the pancreas cancer group as function of the actual level of validation set patients. Prediction threshold (p=0.5) is marked as dashed line.

### Discussion

In this work we developed and validated a method for identifying gastric or pancreatic cancer patients using a simple blood test. Our results are based on models trained and validated on separate data sets.

Based on these data mining results, it is shown that global glycome analysis, for example for analysis of global glycosylation of glycoproteins in serum samples, may be advantageously used to predict cancerous conditions with a relatively high sensitivity and selectivity.

### Appendix

Lectin group composition

| Name | Composition |
|---|---|
| galb1 | RCAI ECL |
| galb2 | RCAI ECL BPL |
| gal_galnac2 | RCAI ECL WGA |
| core0 | GNL HHA |
| core11 | CONA LCA |
| core22 | CONA LCA PSA |
| core33 | CONA LCA PSA GNL HHA |
| core44 | CONA GNL HHA |
| bi1 | LCA PSA |
| bi2 | CONA LCA PSA |
| bi3 | PHAE LCA PSA |
| bi4 | PHAE LCA PSA PVL |
| tri1 | DSA PHAE PHAL |
| sialic1 | MAA SNA |
| sialic2 | MAA SNA SLEA |
| sialic3 | WGA SNA |
| sialic4 | WGA SNA MAA |
| sialic5 | WGA PVL SNA |
| sialic6 | WGA PVL SNA MAA |
| ant44 | RCAI PVL ALAA |
| ant8 | RCAI PVL ALAA SLEA |
| ant9 | RCAI ALAA SLEA |
| ant10 | RCAI ECL SNA MAA |
| ant11 | RCAI ECL SNA WGA PVL MAA |
| ogly3 | ACL PNA DBA |

### Genetic Algorithm Operators and Parameters

Genetic Algorithm (GA) is a general optimization method that uses evolution-based model to minimize (or maximize) a predefined function (called soring function). In the context of GA, the term chromosome is used to describe a mathematical representation of any system or model.
In our case, each chromosome i contains the following data:

n_i, the number of predictors in the chromosome
j_{—}1, j_{—}2, . . . , j_n, predictor indices from a pre-defined list of predictors

1. Initialization.

Seed the population with (about) 100 chromosomes. Call these c_{—}1, . . . , c_{—}100. Each data bit in the chromosome encodes for a single selected parameter. For each chromosome, j, draw n_j (number of predictors in the model) from P_n. Put j into c_j (for j=1 to n_j). Choose the remaining n_j−1 parameters with a uniform random distribution from the remaining parameters.

2. Crossover.

For each two chromosomes C_{—}1 and C_{—}2 that undergo crossover, create chromosome C_{—}3. Choose n_{—}3 (number of predictors in C_{—}3) from {n_{—}1, n_{—}2} each with probability 0.5. Combine the parameters of C_{—}1 and C_{—}2 into a set. Draw n_{—}3 parameters from this set and put them into chromosome C_{—}3.

3. Mutation.

For each chromosome C_i that undergoes mutation, draw n_i* (the new number of predictors in the chromosome) from P_n. if n_i*<n_i, randomly delete elements from C_i until has n_i* elements. If n_i*=n_i then for each element j (j=1 to n_i*) select 0<p<1 from random uniform distribution. If p>P_c (a predefined mutation probability), replace j-th element with a parameter randomly chosen from those not in C_{—}1. If n_{—}1*>n_{—}1 randomly add elements to C_{—}1 until it has size n_{—}1*.
The remaining details of GA are generic and described in [15,16].

### Parameters:

### Generations: 150

Population size: 100
Mutation rate (probability): 0.01
Cross-over rate (probability): 0.9
Convergence epsilon=0.05
Convergence epsilon=20

### Detailed Model Predictions

### Stomach Cancer

TABLE 4
Detailed predictions of stomach cancer model. The sample is
classified as ‘pancreas’ if the predicted probability is above 0.5

| ID | Run | Observed | p_{predicted} |
|---|---|---|---|
| 6019 | 10989 | control | 0.5 |
| 41024 | 10989 | stomach | 0.74 |
| 40996 | 10989 | stomach | 0.98 |
| 6041 | 11007 | control | 0.07 |
| 40972 | 11007 | stomach | 0.89 |
| 30638 | 11007 | stomach | 0.58 |
| 30578 | 11007 | stomach | 0.95 |
| 6022 | 11008 | control | 0.17 |
| 6016 | 11008 | control | 0.19 |
| 30250 | 11008 | stomach | 0.91 |
| 51185 | 11008 | stomach | 0.96 |
| 40650 | 11008 | stomach | 1 |
| 40918 | 11008 | stomach | 0.92 |
| 40906 | 11126 | stomach | 0.98 |
| 30473 | 11126 | stomach | 0.98 |
| 40706 | 11126 | stomach | 1 |
| 41022 | 11205 | stomach | 0.99 |
| 6025 | 11206 | control | 0.08 |
| 6025 | 11548 | control | 0.41 |
| 41022 | 11548 | stomach | 0.56 |
| 30250 | 11550 | stomach | 0.89 |
| 30473 | 11550 | stomach | 0.98 |
| 40650 | 11550 | stomach | 0.86 |
| 6019 | 11551 | control | 0.41 |
| 40706 | 11551 | stomach | 0.9 |
| 6016 | 11552 | control | 0.69 |
| 30578 | 11552 | stomach | 0.96 |
| 6022 | 11568 | control | 0.19 |
| 41024 | 11568 | stomach | 0.38 |
| 40972 | 11568 | stomach | 0.81 |
| 40972 | 11568 | stomach | 0.81 |
| 6041 | 11574 | control | 0.12 |
| 51185 | 11574 | stomach | 0.76 |
| 30638 | 11589 | stomach | 0.51 |
| 40906 | 11589 | stomach | 0.83 |
| 315173 | 12936 | stomach | 0.61 |
| 519311 | 12936 | stomach | 0.52 |
| 176152 | 12937 | stomach | 0.74 |
| 859496 | 12938 | control | 0.37 |
| 199683 | 12938 | stomach | 0.61 |
| 743341 | 12938 | stomach | 0.69 |
| 6.00E+06 | 12940 | control | 0.33 |
| 41009 | 13036 | stomach | 0.91 |
| 51577 | 13036 | stomach | 0.99 |
| 51996 | 13036 | stomach | 0.99 |
| 51708 | 13046 | stomach | 0.72 |
| 51769 | 13046 | stomach | 0.74 |
| 51440 | 13065 | stomach | 0.9 |
| 41009 | 13065 | stomach | 0.68 |
| 30472 | 13091 | stomach | 0.97 |
| 51926 | 13091 | stomach | 0.91 |
| 40709 | 13091 | stomach | 0.86 |
| 51226 | 13091 | stomach | 1 |
| 51484 | 13091 | stomach | 1 |
| 30227 | 13091 | stomach | 0.55 |
| 51577 | 13092 | stomach | 0.84 |
| 40767 | 13092 | stomach | 0.28 |
| 51996 | 13119 | stomach | 0.82 |
| 51406 | 13119 | stomach | 0.64 |
| 30625 | 13119 | stomach | 0.99 |

Pancreas Cancer

TABLE 5
Detailed predictions of pancreas cancer model. The sample is classified
as ‘pancreas’ if the predicted probability is above 0.5

| ID | Run | Observed | p_{predicted} |
|---|---|---|---|
| 6036 | 10989 | control | 0.03 |
| 6026 | 10989 | control | 0.03 |
| 6030 | 11008 | control | 0 |
| 6015 | 11205 | control | 0.04 |
| 6021 | 11205 | control | 0.53 |
| 6028 | 11206 | control | 0.65 |
| 6032 | 11224 | control | 0.02 |
| 30162 | 11548 | pancreas | 0.08 |
| 51315 | 11548 | pancreas | 0.67 |
| 51294 | 11548 | pancreas | 0.9 |
| 51176 | 11550 | pancreas | 0.96 |
| 6032 | 11551 | control | 0.15 |
| 6026 | 11552 | control | 0 |
| 6015 | 11552 | control | 0.02 |
| 40656 | 11552 | pancreas | 0.01 |
| 51861 | 11552 | pancreas | 0.3 |
| 51483 | 11552 | pancreas | 0.06 |
| 6021 | 11568 | control | 0.07 |
| 6030 | 11568 | control | 0.21 |
| 51628 | 11568 | pancreas | 0.99 |
| 6036 | 11589 | control | 0.29 |
| 6028 | 11589 | control | 0.16 |
| 51677 | 11589 | pancreas | 1 |
| 604072 | 12936 | control | 0.88 |
| 609582 | 12937 | control | 0.24 |
| 604072 | 12938 | control | 0.19 |
| 859496 | 12938 | control | 0.23 |
| 51862 | 13036 | pancreas | 1 |
| 62362 | 13055 | pancreas | 0.64 |
| 62410 | 13055 | pancreas | 0.09 |
| 62382 | 13055 | pancreas | 1 |
| 51862 | 13069 | pancreas | 1 |
| 30100 | 13094 | pancreas | 0.92 |
| 51862 | 13094 | pancreas | 0.58 |
| 62248 | 13125 | pancreas | 1 |
| 30057 | 13637 | pancreas | 1 |
| 40950 | 13637 | pancreas | 1 |
| 6030 | 13647 | control | 0 |
| 6021 | 13647 | control | 0 |
| 6032 | 13647 | control | 0 |
| 6026 | 13647 | control | 0 |
| 6036 | 13647 | control | 0 |

### Example 3

### Pancreatic Cancer

This Example relates to glycosylation analysis for the detection of pancreatic cancer. Rather than performing global glycome analysis, this Example demonstrates analysis of a particular protein, haptoglobin, to determine the relationship between its state (or states) of glycosylation and pancreatic cancer in a subject, to determine whether the state (or states) of glycosylation of haptoglobin may be used as a biomarker for diagnosis of pancreatic cancer.

### METHODS

Sample Preparation

In order to test glycosylation of Haptoglobin, a serum fraction enriched with Haptoglobin was prepared as follows: the serum was loaded on Seppro IgY-14 column (GenWay), and the retained 14 most abundant proteins were eluted as described above. This Seppro eluate was depleted from human serum albumin (HSA) and IgG using ProteoSeek Albumin/IgG removal Kit (Pierce PIR-89875) and the mixture of 12 proteins, among them Haptoglobin, was used for glycoanalysis. For calibration standard, a large amount of Seppro IgY-14 eluate from pooled human sera (Sigma) was prepared and pooled.

Glycoanalysis

Slides were pre-wetted and blocked as described for the global glycosylation analysis. The samples (450 ul) were loaded onto the slides at a concentration of 20 ug/ml and incubated for 1 hour on a shaker. Slides were washed as described previously. 450 ul rabbit antibody specific to human Haptoglobin (Dako, A0030) was added at dilution of 1:5000 and incubated for 40 minutes. The slides were washed and detection was done using 450 ul Cy3-labeled anti rabbit antibody (Jackson, 111-765-045) at concentration of 0.75 ug/ml. The slides were incubated for 40 minutes, washed, dried and scanned as described previously. In each experiment a slide for calibration standard sample was included. In addition, a slide to which no sample was applied, served for detecting antibodies background analysis. Signals from this slide were subtracted from all samples signals.

Results

Haptoglobin enriched fraction was prepared from pancreatic cancer patients and healthy control serum samples. To evaluate the applicability of Haptoglobin as biomarker for pancreatic cancer signals from various lectins were subjected to bioinformatic analysis as described in the Computational Methods section of Example 4 below.

### Example 4

### Bioinformatics Glycosylation Analysis for a Particular Serum Protein in Relation to Pancreatic Cancer

This Example relates to the bioinformatics approach used to perform the glycosylation state or states analysis of haptoglobin in Example 3; it is similar to the method used in Example 2 for gastrointestinal cancer (in the Example, stomach cancer), except that the bioinformatics analysis was performed on a single serum protein, haptoglobin, rather than on the global glycome. It should be noted that the biomarkers and methods of use thereof as described in Example 3 are not limited by the particular bioinformatics approach but instead may be used independently of this approach. Similarly, this bioinformatics approach may optionally be used for elucidating any type of cancer biomarker.

The computational methods and data preparation were performed as described for Example 2, as were the lectin group composition, genetic algorithm operators and parameters, as well as the results assessment and validation.

Parameter Selection Process

General Design

The general design is similar to that of global glycosylation profile analysis in Example 2 with the following differences
only pancreas cancer and control patients sera were used
in addition to Bayes and decision tree classifiers, support vector machine (SVM) classifier was added to the parameter selection and training process. This was done to improve the training results. Interestingly, adding SVM to the training phase of global glycosylation analysis resulted in decreased training and validation performances (data not shown)
maximal number of predictors in a model was limited to 3
only the glycosylation of a single protein, haptoglobin, was examined, rather than examining the global glycome

### Results

### Demographic Analysis

Demographic characteristics of the study population are summarized in Table 7. Age distribution is shown in FIG. 7, showing age distribution of the patients in the different study subsets. Box boundaries correspond to the lower and upper quartile values. Horizontal line inside the boxes represents the median. The whiskers show the range of the data.

TABLE 7
Demographic characteristics of the study population. Age is
shown as mean (+/−stdev). Several plasma samples were
glycoprofiled more than once. Number of patients, as well as
the number of glycoprofiles are reported in the table

| | Training | | Validation | |
|---|---|---|---|---|
| | Cancer | Control | Cancer | Control |
| Age | 61.1 (8.4) | 59.8 (11.6) | 61.4 (7.1) | 59.9 (8.9) |
| Patients | 32 | 37 | 20 | 7 |
| out of them | | | | |
| male | 18 | 13 | 7 | 4 |
| female | 14 | 14 | 13 | 3 |

### Data Mining Results

Selecting Attributes

The two best scoring models for pancreatic cancer consist of the following attributes: Model 1: log 2(HPA/bi1); log 2(LCA/HPA); log 2(WFA/gal_galnac2); and Model 2: log 2(WFA/gal_galnac2); log 2(WFA/Siglec-7); log 2(LCA/HPA).

### Predictive Power

Despite the fact that three methods were used during the training process (Bayes classifier, decision trees and SVM), the validation results are reported in term of SVM only, as it consistently produced the best performance. The performance of the best training built with the attributes listed above is detailed in Table 8, along with the corresponding results obtained from the global glycosylation analysis (as reported previously).

Interestingly, despite the fact that the MCC values obtained by analyzing global and single-protein glycoprofiles are pretty similar (0.72 and 0.71, respectively), the sensitivity and specificity of haptoglobin-based model are both higher and more balanced that those obtained with global glycosylation profile. No analysis was performed, as to compare the individual predictions of the two models, as the two studies were performed using different randomization of the data set.

TABLE 8
Performance results for pancreas cancer model.

| | Haptoglobin | | | | Global glycosylation | | | |
|---|---|---|---|---|---|---|---|---|
| Noise (%) | MCC | Sens | Spec | PPV | MCC | Sens | Spec | PPV |
| 0 | 0.71 | 0.8 | 1 | 1 | 0.76 | 0.79 | 0.96 | 0.94 |
| 10 | 0.5 | 0.9 | 0.57 | 0.86 | 0.43 | 0.95 | 0.43 | 0.98 |
| 90 | 0.21 | 0.15 | 1 | 1 | None | 1 | 0 | 0.45 |

Abbreviations are:
CA—accuracy,
MCC—Matthews correlation coefficient,
Sens—sensitivity,
Spec—specificity,
PPV—positive predictive value.
The results of global glycosylation analysis are provided for comparison.
MCC calculations that result in division by zero are marked as “None”.

Detailed model predictions are listed below in Table 9.

Robustness Tests

Re-shuffling the patients' classifications as described in the “Computational methods” section resulted in failure of the training process to generate a model with a reasonable predictive power. The absolute MCC values of the gastric and pancreatic cancer models in the validation set samples did not exceed 0.07, indicating random or near-random classification (data not shown).

As expected, adding a random noise to the validation set data resulted in decreased performance, as measured by MCC: (0.71 vs. 0.50 vs. 0.21 for 0, 10 and 90% noise, respectively.

Model predictions do not depend on the patient cancer stage, as is demonstrated on FIG. 8. FIG. 8 shows the predicted probability of a sample to belong to the pancreas cancer group as function of the actual level of validation set patients. Prediction threshold (p=0.5) is marked as dashed line. Note that due to the fact that SVM classifies samples with p values of either 0.0 or 1.0, the individual data points overlap.

This finding suggests that these models may optionally be used for early cancer detection.

Detailed Model Predictions

Pancreas Cancer

TABLE 9
Detailed predictions of SVM model for the validation set
samples. The sample is classified as ‘pancreas’ if
the predicted probability is above 0.5

| ID | Run | Observed | p_{predicted} |
|---|---|---|---|
| 6016 | 13611 | control | 0 |
| 30057 | 13611 | pancreas | 1 |
| 30162 | 13611 | pancreas | 0 |
| 30612 | 13611 | pancreas | 0 |
| 6021 | 13612 | control | 0 |
| 6026 | 13612 | control | 0 |
| 40701 | 13612 | pancreas | 1 |
| 40892 | 13612 | pancreas | 1 |
| 51681 | 13613 | pancreas | 0 |
| 51308 | 13615 | pancreas | 1 |
| 51315 | 13615 | pancreas | 1 |
| 51567 | 13615 | pancreas | 1 |
| 51629 | 13615 | pancreas | 1 |
| 6028 | 13616 | control | 0 |
| 51292 | 13616 | pancreas | 1 |
| 62382 | 13991 | pancreas | 1 |
| 62410 | 13991 | pancreas | 1 |
| 616452 | 14018 | control | 0 |
| 62314 | 14018 | pancreas | 1 |
| 62252 | 14018 | pancreas | 1 |
| 6090 | 14041 | control | 0 |
| 62248 | 14041 | pancreas | 1 |
| 51862 | 14041 | pancreas | 1 |
| 859546 | 14061 | control | 0 |
| 51628 | 14061 | pancreas | 1 |
| 51861 | 14061 | pancreas | 0 |
| 62380 | 14061 | pancreas | 1 |

### Example 5

### Prostate Cancer

This Example uses a different approach for the detection of prostate cancer, involving precipitating a particular glycosylated protein, PSA, and then analyzing the glycosylation of the precipitated protein.

Various methods were used as described herein.
Method 1—Capture serum PSA with anti-PSA antibody to significantly enrich it and concentrate it prior to analysis by the lectin array platform. Two types of antibodies will be tested in parallel for PSA immunoprecipitation from serum:
   antibodies for total PSA to capture all PSA in the serum
   antibodies specific to free PSA (fPSA, the PSA fraction which is not complexed with ACT and alpha2M) to avoid masking and/or interference by the complexed glycoproteins
Method 2—Increase lectin array platform sensitivity to allow analysis of the low concentrations of the immunoprecipitated PSA
Method 3—Develop an alternative ELISA-based method. This will reduce the number of steps in the assay by direct binding of serum PSA to the anti-PSA-coated plates. It may also allow higher sensitivity.

Lectin array based glycoanalysis was performed as described above.

PSA Immunoprecipitation Assay

Immunoprecipitation of PSA from serum prior to glycoanalysis is preferred due to the low PSA concentrations in serum, and the presence of highly abundant glycoproteins, fat and sugars in the serum which would mask the PSA-specific signals.

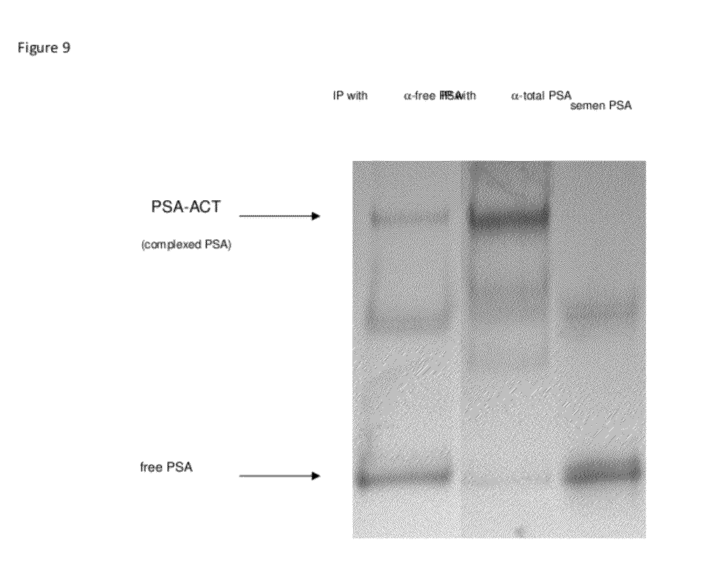

Two methods for immunoprecipitation of PSA from serum were developed, using two different anti-PSA antibodies, for free PSA and for total PSA. The results show that it was possible to immunoprecipitate PSA from prostate cancer patient serum using both methods (FIG. 9).

It was decided to use a monoclonal antibody for free PSA, in order to avoid non-specific signals from ACT and α2M.

However, as can be seen in FIG. 9, although the sample was significantly enriched for free PSA using the anti-free PSA antibody, some complexed PSA was pulled down as well (ACT-PSA), showing that the antibody is not entirely specific for free PSA.

Since it is estimated that analysis of total PSA would introduce non-specific signals from ACT and α2M, more efforts are now directed towards separation of the free PSA from the complexed PSA. Three approaches are being explored in parallel:
separation of free PSA from complexed PSA on gels, and extracting the free PSA band by electro-elution
separation of free PSA from complexed PSA using immunodepletion of complexed PSA using anti-ACT antibody-coated beads
testing additional monoclonal antibodies for free PSA

Successfully separated free PSA is analyzed by the lectin array.

### Increase Lectin Array Platform Sensitivity

The current sensitivity for PSA glycoanalysis assay is around 300 ng/ml (60 ng/slide). This sensitivity allows analysis of only the higher PSA samples from prostate cancer patients, but not of the benign hyperplasia samples and the samples from healthy individuals. Additional increase in the sensitivity is required.

To achieve this the following potential methods are being considered:
Reduce cross interactions between the arrayed lectins and the anti-PSA antibody (probe). This will allow better sample/control ratio
   Modify anti-PSA antibody (PNGase etc.)
   Test additional anti-PSA antibodies as probes
   Different blocking options
Enhance signals by signal amplification using streptavidin-FITC on top of the secondary (biotinylated) anti-IgG antibody
Change fluorescent dye to a brighter dye (FITC is relatively weak)
Reduce background by different slide types (2D)
Increase detection sensitivity by using Evanescent field scanner

### Alternative Systems—ELISA

Analysis of PSA samples on anti-PSA-coated ELISA plates using lectins as probes may optionally be performed. This method is suggested to increase efficiency of the assay as well as sensitivity. It is also more applicable to current laboratory equipment in diagnostics labs.

### Example 6

### Glycodiagnostics II, Slide Preparation

This Example relates to global glycome analysis for the detection of cancer, through an improved detection process. For this non-limiting example (and corresponding optional embodiments of the present invention), epoxy slides were used, as opposed to the nitrocellulose coated slides of the above Examples. These slides significantly reduce the background and hence increase the sensitivity of the diagnostic method.

Materials and Methods

General Study Design

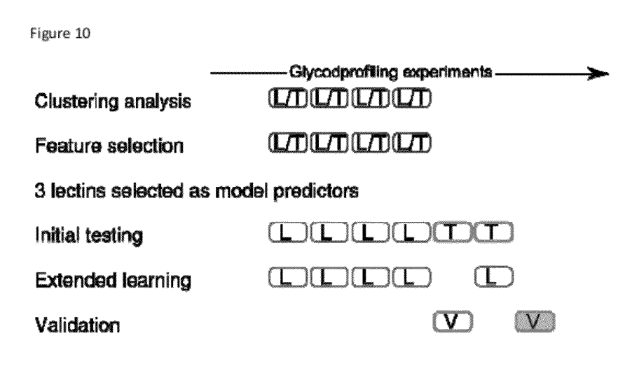

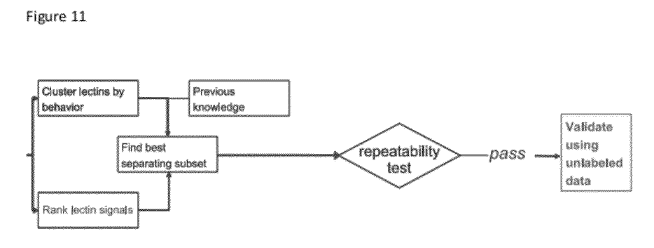

The study design is schematically summarized by FIGS. 10 and 11. Initial portion of glycoprofile experiments was performed and subjected to bioinformatic analysis. The bioinformatic methods applied in this study are slightly different from those described in Examples 2 and 4. The detailed description of bioinformatic methods, as well as classification results, are listed below as Example 7. Based on these analyses, a set of three selected lectins was derived, as non-limiting examples only of a set of lectins; clearly any plurality of lectins could optionally be used with these non-limiting embodiments of the present invention. The results of the binding behavior of these three lectins in all the experiments available were used to train a naïve Bayesian classifier. The next step was performing another set of glycoprofiling experiments with sera that were not analyzed yet and testing the classifier on the resulting signals. In order further improve the predictive abilities of the model, and as a new stage that was not previously used for the above Examples, it was decided to expand the training data of the Bayesian classifier with more glycoprofile signals (the “extended learning” phase in the diagram on FIG. 10). The generalization capacity of the resulting model was tested using additional previously unseen samples.

The overall design of the study described in this EXAMPLE is as follows:
GMID readings of all the lectins in every serum sample are obtained
Raw lectin signals are compared to the respective signals in control standard (relative signals)
The lectins are clustered by the correlation between their relative signals in all the test samples
In parallel to this clustering, statistical ranking of the lectins is performed
The clustering data, combined with the ranking of the relative lectin signals and with previous knowledge on glycan epitopes that are expected to change in cancer conditions are all combined to suggest set of possible separators
The models that are created with the candidate sets of separating lectins are subjected to repeatability and robustness tests and the best performing model is validated using previously unseen and unlabeled results

This process is schematically depicted in FIG. 11. The flow of test samples over the study is schematically depicted by FIG. 10. Each glycoprofiling experiment is represented by a box. The letters inside the boxes represent learning samples (L), testing samples (T) and validation samples (V). The vertical coordinate in FIG. 10 represents study time. It should be emphasized that the GMID signals of the validation samples were not available during the training process and were obtained only after the training has been completed.

As shown in FIG. 11, the lectin signals were ranked and hierarchically clustered using correlation between the signals as the distance function. It was verified that the obtained clusters are corroborated by the existing literature data (e.g lectins that are specific to similar sugar epitopes are located close to each other in the clustering hierarchy). The best separating subset of lectins was located. This search was done by selecting the set of lectins that were most diagnostically discriminatory for the particular cancer, but do not reside too close (less than two junctions) in the clustering hierarchy. The best separating subset was then applied to sera that had not been previously tested, to confirm the diagnostic utility of the subset. This step is termed as “initial testing” on FIG. 10. Next, half of the initial testing samples were added to the training set and new classification model was constructed using the same lectin group (“extended learning” on FIG. 10). The purpose of this step was to increase the robustness of the model. The resulting model was tested on the remaining half of the “initial testing” samples and on additional, previously unseen, set of samples (“validation” on FIG. 10).

Methods—Biological Assay

Serum samples (identical samples to those of Example 1) that were used in this Example were of Caucasian patients that are “drug naïve”, for which comprehensive demographic and clinical data were available. The samples were supplied by two different sources: RNTECH (France) and Asterand (USA).

Sample Preparation

Serum samples were depleted of 14 most abundant proteins using IgY-14 spin column (Sigma), according to manufacturer instructions. At all stages 1×PBS was used instead of the Tris-HCL buffer recommended by the manufacturer. Basically, 15 ul of serum was diluted in 1×PBS to final volume of 500 ul. The diluted serum was filtered by Spin-X (Costar) and the strained serum was loaded onto the depletion IgY-14 column. The unbound fraction (depleted serum) was collected. The bound proteins were eluted and neutralized using the kit stripping and neutralization buffers. The column was regenerated for further use (up to 100 times).

The depleted serum was fluorescently labeled (final Fluorophor/Protein=1) using Cy3—NHS (Amersham). Following incubation for 2 hours at 4° C. on an end-over-end shaker the reaction was stopped with 100 ul of 1M Tris-HCl pH 7.5 per 1 ml. MiniTrap G-25 columns (GE Healthcare), equilibrated with 10 ml with PBS, were used to separate free Cy3 from labeled sample.

Glycoanalysis

The lectin arrays used were comprised of epoxysilane coated glass slides (Schott) printed with various lectins from different sources such as plant, human, etc, as well as antibodies to glycan epitopes. Each slide was printed with 7 identical lectin arrays to allow simultaneous and high throughput analysis of multiple samples. At least three slides were processed simultaneously for the analysis of one CS (control) and 20 actual samples. The minimal experiment requires one CS and one sample. The required solutions and volumes for the amount of slides processed in the experiment were prepared. Cy3-labeled depleted serum sample was prepared in wash buffer containing 1×PBS, 0.4 mM MgCl₂, 0.4 mM CaCl₂, 0.004M MnCl₂, 0.05% Tween 20 to a final protein concentration of Bug/ml.

Procedure

A multi-pad incubation frame (GraceBio) was adhered onto each slide printed with identical lectin arrays. Lectin arrays were handled carefully, wearing non-powdered gloves during slide handling and avoiding any contact with the lectin printed surface.

Arrays were incubated with Cy3-labeled samples in the dark on an orbital shaker set to rotate at 50 rpm over night (17 h) at room temperature (15-25° C.). The slides were kept covered at all times to minimize evaporation and light in order to prevent drying out of slides and bleaching of fluorescence. At the end of the incubation the arrays were washed twice in the dark and the incubation frames were carefully removed. Slides were washed with 25 ml RO- or HPLC-grade water, and dried. The arrays were scanned and analyzed.

Drying Slides after Processing

To avoid nonspecific background signals, slides were dried before scanning.

Slide(s) were removed from final water wash, and centrifuged at 200×g for 5-10 min (or until slides are dry) in a Coplin jar or a centrifuge slide carrier, then air dried in the dark until the membrane was completely white.

Scanning Slides

Following sample processing and drying, slides were scanned using a microarray Laser scanner with adjustable laser power and photomultiplier tube (PMT), (Axon GenePix 4200) with Cy3 filter. Images were analyzed using image analysis software: Array-Pro™ ver. 4.5 (Media Cybernetics, Inc) and PPIP ver 2.0 (Procognia Proprietary Image Processing, Procognia, Ltd).

Results

In this project, the use of microarrays for glycoanalysis of purified glycoproteins was expanded to the analysis of complex protein mixtures from serum. Untreated serum contains about 30-50 mg/ml of albumin and IgG. To enable detection of lower abundance serum proteins a method for removal of 14 most abundant serum/plasma proteins by a mixed antibodies commercial antibody resin (Seppro IgY-14, Sigma) was established. The antibodies of the resin were directed against HSA, IgG, Fibrinogen, Transferrin, IgA, IgM, Apo A-I, Apo A-II, Haptoglobin, alpha-1-Antitripsin, alpha-1-Acid Glycoprotein, alpha-2-Macroglobulin, complement C3 and LDL. In addition a method for Cy3 labeling of serum proteins and dye clean-up was calibrated. Finally, optimal protein concentration for analysis on the lectin microarray was determined and the entire processing protocol was established.

The lectin array consists of a set of 35 lectins printed on a epoxysilane-coated glass slide. When the sample of intact purified glycoprotein is applied to the array, and its binding pattern is detected by direct labeling using fluorophore, the resulting fingerprints are highly characteristic of the glycosylation pattern of the sample. The large number of lectins, each with its specific recognition pattern, ensured high sensitivity of the fingerprint to changes in the glycosylation pattern.

### Example 7

### Bioinformatics Glycome Analysis II

This Example relates to the bioinformatics approach used for global glycome analysis in Example 6, using epoxy slides. It should be noted that the biomarkers and methods of use thereof as described in Example 6 are not limited by the particular bioinformatics approach but instead may be used independently of this approach. Similarly, this bioinformatics approach may optionally be used for elucidating any type of cancer biomarker through global analysis of the glycome.

Data Analysis and Parameter Selection

Two types of serum samples were analyzed during each glycoprofile experiment: a calibration standard (CS) and a test sample. The CS sample was obtained from a serum of a single healthy volunteer. This sample served as a reference point for the test samples in different experiments.

The input of all the data analysis techniques is the log ratio between the test sample signal of a certain lectin to that of the same lectin in the CS sample. In order to avoid division by zero error, a constant value is added to both nominator and denominator, as follows:

$${x}_{i} = log ( \frac{{CS}_{i} + 1}{{T}_{i} + 1} ) ,$$

where CSᵢ is the i-th lectin signal in the CS sample and Tᵢ is the i-th lectin signal in the test sample in the same experiment.

The next step for this Example was to select a small subset of lectins that would generate best model for distinguishing between cancer and cancer free subject. Theoretically, a simple ranking technique (such as chi-square ranking) could have been used to select top ranking lectins and build the model upon them; this strategy, often termed as greedy, is an option according to some embodiments of the present invention. However, in the case discussed here, there is a high correlation between the signals resulting from binding of several lectin groups. When such a correlation is present, the greedy optimization approach has been proven to be non-effective and prone to overfitting. Therefore, a different strategy was used for this Example, as described below.

In order to identify those lectins that can result in the best predictive models, the resulting relative lectin signals (xᵢ in the equation above) were subjected to three levels of analysis: (i) lectin ranking; (ii) hierarchical clustering and (iii) literature analysis.

Lectin ranking. A chi-square feature selection method was used to rank the lectins based on their relative signals and on the actual sera labeling.

Hierarchical analysis. The purpose of hierarchical analysis of relative lectin signals is to identify common patterns of lectin behavior irrespective with the source of the tested serum (cancer or control group). A simple Pearson correlation coefficient was used as the distance metric supplied to the clustering algorithm. Lectins that are clustered close one to another are considered to present similar behavior in glycoprofile experiments.

Literature analysis. Literature analysis is a supplementary method to support the selection of a subset of lectins that are corroborated by the existing published scientific results on glycosylation aberrations in cancer.

The three levels of analysis were combined to select subsets of lectins that are (i) predictive to cancer/control classification; (ii) present as few colinearities as possible and (iii) are corroborated by existing biological knowledge where possible. As a result, several (˜5) alternative lectin subsets were constructed and tested with different randomization divisions of the learning/testing examples (first two rows in FIG. 10). The best performing lectin set was then carried out to the remaining steps.

Note the difference between the parameter selection process described here and the one described in Example 2. The method described in Example 2 is an optimization process that scans a huge hyperspace of possible models. The method described here results in testing of not more than half a dozen alternative models.

Results

Demographic Analysis

The demographic properties of the study population are summarized in Table 6.

TABLE 6
Demographic properties of study population

| | Training | Validation |
|---|---|---|
| Patients | 86 | 58 |
| out of them | | |
| female | 50 | 19 |
| male | 36 | 39 |
| stomach | 38 | 27 |
| control | 48 | 31 |
| Age: mean (±std) | | |
| total | 57.8 (10.8) | 61.5 (10.5) |
| stomach | 63.2 (9.1) | 64.7 (8.8) |
| control | 53.6 (10.3) | 57.6 (11.2) |

The Selected Model and its Performance

The following lectins were selected for the model PVL, PSA, Anti-sLeA. Model performance in the extended learning and in the validation data sets are summarized in Table 7.

TABLE 7
Accuracy (CA), Matthews's correlation coefficient (MCC),
sensitivity (Sens) and specificity (Spec) for the naïve Bayes
classifier in the study population samples

| | CA | MCC | Sens | Spec |
|---|---|---|---|---|
| Learning | 0.72 | 0.42 | 0.64 | 0.77 |
| Validation | 0.72 | 0.45 | 0.7 | 0.74 |

### Example 8

### Glycodiagnostics Through Hydrogel Coated Slides

This Example relates to another glycodiagnostic method, involving polymer (hydrogel) coated slides according to other optional, non-limiting embodiments of the present invention.

### Methods

Study Design Overview

Following is a brief description of the study design. It will be discussed in detail in the following sections
Serum samples are depleted from 14 most abundant proteins by the means of immunoaffinity
Depletion process is done in batches of 16 samples in parallel, although other sample sizes (for example 96 samples) could optionally be used.
There are four types of serum samples in this study:
   Healthy volunteers
   Gastric cancer patients
   External calibration standard (ECS)—a sample from a healthy volunteer that has been obtained and depleted in a large quantity
   Internal calibration standard (CS)—a sample from the same healthy volunteer obtained in a large quantity, but depleted separately in each depletion batch
Note that the ECS samples serve for quality assurance purposes and so the results obtained from these samples were not included in the classification.
The serum samples are applied on SurModics Hydrogel slides (8 samples on each slide) and are subjected to GMID assay (2 slides per assay). Each GMID assay contains 14 serum samples, one internal and one external calibration standard samples.
There are four sets of GMID data in this study:
   Training-I—a set of serum samples, in addition to ECS and CS. This set serves to select model predictors and model training
   Training-II—same samples as in Training-I. The models developed with the Training-I set are tested with Training-II. Afterwards, the corresponding relative lectin signals were averaged to create more robust models
   Validation-I and Validation II—a set of serum samples not included in the Training sets, in addition to ECS and CS. These samples were analyzed twice in different GMID experiments. These sets are used to blindly validate the predictive model developed in the previous steps.
      More detailed description of data handling appears in Example 8.

Biological Assay

The serum samples used were of Caucasian patients that are “drug naïve”, for which comprehensive demographic and clinical data were available, and were the same samples as for Examples 1 and 6. The samples were supplied by two different sources: RNTECH (France) and Asterand (USA).

### Sample Preparation

Serum samples were depleted of 14 most abundant proteins using a commercial antibody resin (Seppro IgY-14, Sigma), according to manufacturer instructions. At all stages 1×PBS was used instead of the Tris-HCL buffer recommended by the manufacturer. Basically, 15 ul of serum was diluted in 1×PBS to final volume of 500 ul. The diluted serum was filtered by Spin-X (Costar) and the strained serum was loaded onto the depletion IgY-14 column. The unbound fraction (depleted serum) was collected. The bound proteins were eluted and neutralized using the kit stripping and neutralization buffers (Seppro® IgY14, Sigma). The column was regenerated for further use (up to 100 times).

The depleted serum was fluorescently labeled (final Fluorophor/Protein=1) using Cy3—NHS (Amersham). Following incubation for 2 hours at 4° C. on an end-over-end shaker the reaction was stopped with 100 ul of 1M Tris-HCl pH 7.5 per 1 ml. MiniTrap G-25 columns (GE Healthcare), equilibrated with 10 ml with PBS, were used to separate free Cy3 from labeled sample.

### Glycoanalysis

The lectin arrays used were comprised of hydrogel coated glass slides (HD slides) containing N-hydroxysuccinimide ester groups (Surmodics) and printed with various lectins from different sources such as plant, human, etc, as well as antibodies to glycan epitopes. Each slide was printed with 8 identical lectin arrays to allow simultaneous and high throughput analysis of multiple samples. The required solutions and volumes for the amount of slides processed in the experiment were prepared. Cy3-labeled depleted serum sample was prepared to a final protein concentration of Bug/ml in complete wash buffer containing 1×PBS, 0.4 mM MgCl₂, 0.4 mM CaCl₂, 0.004M MnCl₂, 0.05% Tween20.

### Procedure

A multi-pad incubation frame (GraceBio) was adhered onto each slide printed with identical lectin arrays. Lectin arrays were handled carefully, wearing non-powdered gloves during slide handling and avoiding any contact with the lectin printed surface.

Pre-wetting of the slides was performed using complete wash buffer containing, PBS×1, 0.4 mM MgCl2, 0.4 mM CaCl2, 0.004M MnCl2 and 0.05% Tween20. Pre-wetting solution was removed and 0.15 ml complete blocking solution containing 1×PBS, 0.4 mM MgCl2, 0.4 mM CaCl2, 0.004M MnCl2, 1% BSA, 0.05% Tween20 was added to each array, which was then incubated on an orbital shaker set to rotate at 50 rpm for 60 min at room temperature (15-25° C.). Blocking solution was discarded.

Arrays were washed three times with 0.2 ml complete wash solution. After the third wash step, 150 μl sample was gently pipetted onto each lectin array, ensuring that the array is fully covered, without touching the array, and avoiding formation of bubbles. The procedure was repeated for the remaining arrays.

Arrays were incubated in the dark on an orbital shaker set to rotate at 50 rpm over night (17 h) at room temperature (15-25° C.). The slides were kept covered at all times to minimize evaporation and light, in order to prevent drying out of slides and bleaching of fluorescence.

Arrays were washed twice in the dark by adding 0.2 ml complete wash solution to each array. The incubation chambers were carefully removed and slides were washed in the dark two more times in complete wash solution and once in RO- or HPLC-grade water (25 ml per slide), and dried. The arrays were scanned and analyzed.

Drying Slides after Processing

To avoid nonspecific background signals, slides were dried before scanning.

Slide(s) were removed from final water wash, and centrifuged at 200×g for 5-10 min (or until slides are dry) in a Coplin jar or a centrifuge slide carrier, then air dried in the dark until membrane is completely white.

### Scanning Slides

Following sample processing and drying, slides were scanned using a microarray Laser scanner with adjustable laser power and photomultiplier tube (PMT), (Axon GenePix 4200) with Cy3 filter. Images were analyzed using image analysis software: Array-Pro™ ver. 4.5 (Media Cybernetics, Inc) and PPIP ver 2.0 (Procognia Proprietary Image Processing, Procognia, Ltd).

Results

In this project, the use of microarrays for glycoanalysis of purified glycoproteins was expanded to the analysis of complex protein mixtures from serum. Untreated serum contains about 30-50 mg/ml of albumin and IgG. To enable detection of lower abundance serum proteins a method for removal of 14 most abundant serum/plasma proteins by a mixed antibodies commercial antibody resin (Seppro IgY-14, Sigma) was established. The antibodies of the resin are directed against HSA, IgG, Fibrinogen, Transferrin, IgA, IgM, Apo A-I, Apo A-II, Haptoglobin, alpha-1-Antitripsin, alpha-1-Acid Glycoprotein, alpha-2-Macroglobulin, complement C3 and LDL. In addition a method for Cy3 labeling of serum proteins and dye clean-up was calibrated. Finally, optimal protein concentration for analysis on the lectin microarray was determined and the entire processing protocol was established.

The lectin array consists of a set of 40 lectins printed on a polymer-coated glass slide. When sample of intact purified glycoprotein is applied to the array, and its binding pattern is detected by direct labeling using fluorophore, the resulting fingerprints are highly characteristic of the glycosylation pattern of the sample. The large number of lectins, each with its specific recognition pattern, ensures high sensitivity of the fingerprint to changes in the glycosylation pattern. It should be noted that this Example uses a greater number of lectins than previous Examples; without wishing to be limited by a single hypothesis, it is believed that the increased number of lectins may lead to greater sensitivity and/or specificity.

### Example 9

### Bioinformatics Glycome Analysis III

This Example relates to the bioinformatics approach used for global glycome analysis in Example 8, using polymer coated slides. It should be noted that the biomarkers and methods of use thereof as described in Example 8 are not limited by the particular bioinformatics approach but instead may be used independently of this approach. Similarly, this bioinformatics approach may optionally be used for elucidating any type of cancer biomarker through global analysis of the glycome.

Bioinformatics Methods

Data Normalization and Standardization

The lectin array was applied on SurModix slides as described in the previous Example in batches of slides, termed as “printings”. In order to account for the differences between glycosylation pattern signals in slides from different printing batches, the raw signals were standardized as follows (Wang, J., et al. (2007)):
signal intensity was log (base 2) transformed
standardized signal was calculated as follows:

$${g}_{i , s} = \frac{{X}_{i , s} - \overline{{X}_{i , ( control )}}}{{σ}_{i , ( control )}}$$

Where g_{i,S} denotes the standardized signal of lectin i in sample S; X_{i,S} is the log₂ intensity of that lectin before the standardization, normalized to the sum of sample signals; X̅_̅{̅i̅,̅(̅c̅o̅n̅t̅r̅o̅l̅)̅}̅ and σ_{i,(control)} are respectively, the average signal intensity and the standard deviation of calibration standard (CS) in the same printing batch. In order to avoid division by zero, if only one reference sample is available σ_{i,(control)} is arbitrarily set to 1. Duplicate relative signals (g_{i,S}) were average and these average values were used to either train or test the classification models

Lectin Groups

Based on similar specificity patterns, the following lectin groups have been identified: Fucose and Sialic Acid. In this Example, the Fucose group consists of relative signals of UEAI and AOL, while Sialic Acid group consists of signals of Siglec-5 and Siglec-7. The corresponding signals are subjected to principal component analysis (PCA) and the first component is taken as the group representative. Lectin signals and group signals are collectively termed as “predictors”.

Classification Method and Predictor Selection

In this Example, Logistic regression was used to classify the sample serums. Logistic regression is a generalized linear model used for binomial regression. This model is described by a linear combination of coefficients as follows:

Fₛ=β₀+β₁g_{1,S}+ . . . +βₙg_{n,S},

where β₀ is the intercept; and βᵢ is the i-th coefficient. The probability, p_{S} of a sample S to be considered as “cancer” is computed as:

$${p}_{s} = \frac{{e}^{{F}_{s}}}{1 + {e}^{{F}_{s}}} .$$

A sample is classified as “cancer” if p_{S} is equal or greater than a certain threshold (0.5 in this Example). Otherwise the sample is classified as “control”. The training set samples are used to estimate the values of coefficients. It is also possible to compute the statistical probability, p_{β,i}, that the coefficient βᵢ is significantly different from zero.

In order to identify classification models, the training set data was used to exhaustively scan all possible combinations of three (out of all available) predictors. A logistic regression model was created using each such combination. Each model was evaluated using MCC of 10-folds cross validation. Next, 15 models with highest MCC values were examined, filtering out those models in which p_{β,i} for each βᵢ is greater than 0.1. The predictor set identified in Example 7 (PVL, PSA, Anti-dLeA) was also tested.

### Results

Demographic Analysis

Demographic properties of the study population of this Example are summarized in Table 8.

TABLE 8
Demographic analysis of the patients participating in Example 9

| | Training | Validation |
|---|---|---|
| Patients | 91 | 80 |
| out of them | | |
| female | 37 | 35 |
| male | 54 | 45 |
| stomach | 38 | 35 |
| control | 53 | 45 |
| Age: mean (±std) | | |
| total | 59.2 (9.9) | 59.4 (11.6) |
| stomach | 63.4 (7.3) | 65.6 (8.7) |
| control | 56.1 (10.4) | 54.5 (11.3) |

Predictor Selection

Five sets of predictors were identified as described in the Methods section of this Example. The results of the models built with these predictor sets are summarized in Table 9 and are sorted by the MCC value of the training. In addition to the five predictor sets, the results for predictor set that was identified in Example 7 are also reported.

TABLE 9
Classification of Logistic regression models built with the data
of Example 9. The first column describes the predictors that were
used to create the corresponding model

| | Training | | | | Validation | | | |
|---|---|---|---|---|---|---|---|---|
| | CA | MCC | Sens | Spec | CA | MCC | Sens | Spec |
| STL, ALAA, Sialic | 0.9 | 0.86 | 0.92 | 0.94 | 0.8 | 0.65 | 0.75 | 0.89 |
| acid group | | | | | | | | |
| ECL, ALAA, | 0.9 | 0.82 | 0.84 | 0.96 | 0.8 | 0.6 | 0.71 | 0.88 |
| DC-SIGN | | | | | | | | |
| DSA, ALAA, | 0.9 | 0.8 | 0.87 | 0.92 | 0.8 | 0.63 | 0.75 | 0.87 |
| DC-SIGN | | | | | | | | |
| ALAA, DC-SIGN, | 0.9 | 0.77 | 0.84 | 0.92 | 0.8 | 0.6 | 0.75 | 0.85 |
| Siglec-5 | | | | | | | | |
| ALAA, Siglec-5, | 0.9 | 0.75 | 0.82 | 0.92 | 0.8 | 0.64 | 0.77 | 0.87 |
| Fucose group | | | | | | | | |
| PVL, PSA, | 0.8 | 0.59 | 0.68 | 0.89 | 0.7 | 0.37 | 0.53 | 0.82 |
| Anti-sLeA | | | | | | | | |

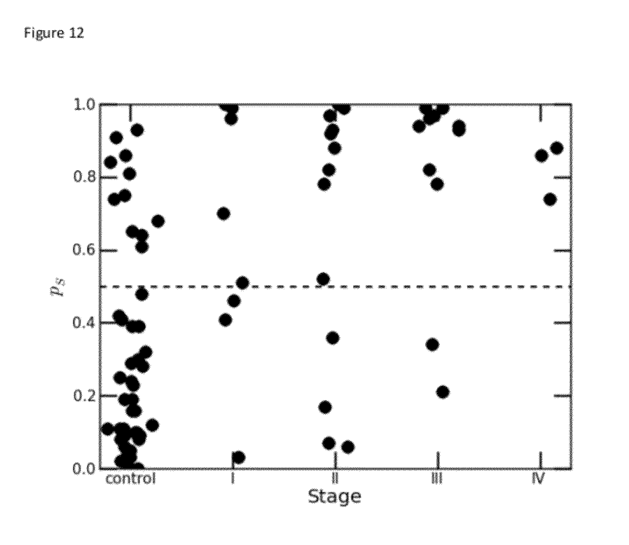

As stated above, the output logistic regression classifier (among others) is the probability, p_{S}, that a sample S belongs to the stomach cancer group. The sample is considered as positive (cancer) if p_{S} is 0.5 or above. FIG. 12 shows the predicted values of p_{S} predicted by the model based on ALAA, Siglec-5 and Fucose group as a function of the disease stage of the validation set patients. The classification threshold of 0.5 is depicted by a dashed horizontal line.

As noted above, the term “fucose group” features binding of UEAI and AOL; while the term “sialic acid group” features binding of Siglec-5 and Siglec-7; as the saccharide binding agents, respectively.

### REFERENCES

Kim, Y. J. and Varki, A. (1997) Perspectives on the significance of altered glycosylation of glycoproteins in cancer. Glycoconj. J., 14, 569-576.
Dube D H and BErtozzi C R, Glycans in cancer and inflammation—potential for therapeutics and diagnostics. Nat Rev Drug Discov. 2005 June; 4 (6):477-88
Kobota, A and Amano, J. Altered glycosylation of proteins produced by malignant cells, and application for the diagnosis and immunotherapy of tumours. Immunol Cell Biol. 2005 August; 83 (4):429-39. Review.
Goggins, M., Molecular markers of early pancreatic cancer. J Clin Oncol. 2005 Jul. 10; 23 (20):4524-31.
DiMagno E. P., Reber H. A., Tempero M. A. Epidemiology, diagnosis, and treatment of pancreatic ductal adenocarcinoma. Gastroenterology, 117: 1463-1484, 1999
Jemal A., Murray T., Samuels A., Ghafoor A., Ward E., Thun M. J. Cancer statistics 2003. CA Cancer J. Clin., 53: 5-26, 2003
Yeo C. J., Cameron J. L., Lillemoe K. D., Sitzmann J. V. Pancreaticoduodenectomy for cancer of the head of the pancreas: 201 patients. Ann Surg., 221: 721-731, 1995
Pisani, P., Parkin, D. M., Bray, F., Ferlay, J., Estimates of the worldwide mortality from 25 cancers in 1990. Int. J. Cancer, 8, 18-29, 1999.
Lam, K. W., and S. C., Lo. Discovery of diagnostic serum biomarkers of gastric cancer using proteomics. Proteomics Clin. Appl., 2, 219-228, 2008. Review
Peracaula R, Tabares G, Royle L, Harvey D J, Dwek R A, Rudd P M, de Llorens R. Altered glycosylation pattern allows the distinction between prostate-specific antigen (PSA) from normal and tumor origins. Glycobiology. 13: 457-70. 2003
Peracaula et al. Glycosylation of human pancreatic ribonuclease: differences between normal and tumor states. Glycobiology. 13 (4):227-44, 2003a.
Hamid U. M. A. et al. A strategy to reveal potential glycan markers from serum glycoproteins associated with breast cancer progression. Glycobiology. 18 (12): 1105-1118, 2008.
[1] A. I. Neugut, M. Hayek, G. Howe, Epidemiology of gastric cancer. Seminars In Oncology. 23 (1996) 281-91.
[2] D. M. Parkin, Epidemiology of cancer: global patterns and trends. Toxicology Letters. 102-103 (1998) 227-34.
[3] L. Ries, C. Kosary, B. Hankey, B. Miller, . . . , SEER cancer statistics review, 1973-1996, Bethesda, Md.: National Cancer Institute. (1999).
[4] M. P. Coleman, J. Esteve, P. Damiecki, A. Arslan, H. Renard, Trends in cancer incidence and mortality, Cancer Causes & Control. 5 (1994) 293-293.
[5] J. Forgensen, Resected adenocarcinoma of the pancreas—616 patients: results, outcomes, and prognostic indicators. Journal Of Gastrointestinal Surgery: Official Journal Of The Society For Surgery Of The Alimentary Tract. 5 (n.d.) 681; author reply 681.
[6] A. Jemal, R. C. Tiwari, T. Murray, A. Ghafoor, A. Samuels, E. Ward, et al., Cancer statistics, 2004. CA: A Cancer Journal For Clinicians. 54 (n.d.) 8-29.
[7] A. Jemal, R. Siegel, E. Ward, Y. Hao, J. Xu, M. J. Thun, Cancer statistics, 2009. CA: A Cancer Journal For Clinicians. 59 (n.d.) 225-49.
[8] M. Feldman, M. Sleisenger, B. Scharschmidt, Tumors of the stomach, in: Feldman M, Sleisenger And Fordtran's Gastrointestinal And Liver Disease: Pathophysiology, Diagnosis, Management, 8 ed., Saunders, n.d.p. 257.
[9] F. Faggiano, T. Partanen, M. Kogevinas, P. Boffetta, Socioeconomic differences in cancer incidence and mortality. IARC Scientific Publications. (1997) 65-176.
[10] F. Kitahara, K. Kobayashi, T. Sato, Y. Kojima, T. Araki, M. A. Fujino, Accuracy of screening for gastric cancer using serum pepsinogen concentrations. Gut. 44 (1999) 693-7.
[11] B. D. Westerveld, G. Pals, C. B. Lamers, J. Defize, J. C. Pronk, R. R. Frants, et al., Clinical significance of pepsinogen A isozymogens, serum pepsinogen A and C levels, and serum gastrin levels. Cancer. 59 (1987) 952-8.
[12] G. D. Smith, C. Hart, D. Blane, D. Hole, Adverse socioeconomic conditions in childhood and cause specific adult mortality: prospective observational study. BMJ (Clinical Research Ed.). 316 (1998) 1631-5.
[13] A. J. van Loon, R. A. Goldbohm, P. A. van den Brandt, Socioeconomic status and stomach cancer incidence in men: results from The Netherlands Cohort Study. Journal Of Epidemiology And Community Health. 52 (1998) 166-71.
[14] B. W. Matthews, Comparison of the predicted and observed secondary structure of T4 phage lysozyme. Biochimica Et Biophysica Acta. 405 (1975) 442-51.
[15] A. Butterfield, V. Vedagiri, E. Lang, C. Lawrence, M. J. Wakefield, A. Isaev, et al., PyEvolve: a toolkit for statistical modelling of molecular evolution. BMC Bioinformatics. 5 (2004) 1.
[16] D. E. Goldberg, Genetic Algorithms in Search, Optimization, and Machine Learning, Addison-Wesley Professional, n.d.
J. N. Arnold, R. Saldova, U. M. Abd Hamid and P. M. Rudd. Evaluation of the serum N-linked glycome for the diagnosis of cancer and chronic inflammation. Proteomics, 2008, 8, 3284-3293.
Taketa K. et al. 1993. A collaborative study for the evaluation of lectin-reactive alphafetoproteins in early detection of hepatocellular carcinoma. Cancer Res. 53: 5419-5423.
Okuyama N. Et al. 2006. Fucosylated haptoglobin is a novel marker for pancreatic cancer: A detailed analysis of oligosaccharide structure and a possible mechanism for fucosylation. Int. J. Cancer Vol. 118 PP. 2803-8.
Miyoshi E. and M. Nakano. 2008. Fucosylated Haptoglobin is a novel marker for pancreatic cancer: Detailed analysis of oligosaccharide structures. Proteomics. 8: 3257-3262.
Thompson S. and G. A. Turner. 1987. Elevated levels of abnormally-fucosylated haptoglobins in cancer sera. Br. J. Cancer. 56:605-610.
Turner G. A. 1995. Haptoglobin. A potential reporter molecule for glycosylation changes in diseases. Adv. Exp. Med. Biol. 376: 231-238.
Nakano M. et al. 2008. Site specific analysis of N-glycans on Haptoglobin in sera of patients with pancreatic cancer: A novel approach for the development of tumor markers. Int. J. Cancer. 122: 2301-2309.
Wang, J., et al. (2007) Merging microarray data, robust feature selection, and predicting prognosis in prostate cancer. Cancer informatics, 2, 87-97.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Citation or identification of any reference in this application shall not be construed as an admission that such reference is available as prior art to the present invention.

To the extent that section headings are used, they should not be construed as necessarily limiting.

1. A biomarker for detecting stomach cancer in a sample taken from a subject, comprising one or more glycans having reactivity to one or more of the following saccharide binding agent combinations: HHA and Anti-sLeA; PSA and bi3; bi2 and bi4; DSA and HPA; STL, ALAA, and Sialic acid group; ECL, ALAA, and DC-SIGN; DSA, ALAA, and DC-SIGN; ALAA, DC-SIGN, and Siglec-5; ALAA, Siglec-5, and Fucose group; or PVL, PSA, and Anti-sLeA; or a combination or a ratio thereof.

2. The biomarker of claim 1, wherein the biomarkers are selected from the following analytical biomarker functions: Model 1-log 2(HHA/Anti-sLeA); Log 2 PSA/Log 2 bi3; log 2(bi2/bi4); log 2(DSA/HPA); and Model 2-log 2(bi2/bi4); log 2(PSA/bi3); log 2(HHA/Anti-sLeA).

3. The biomarker of claim 2, wherein said glycan comprises a motif selected from the group consisting of Fucose, Sialyl Lewis A; High mannose, Bi-antennary; Tri/tetra-antennary; High mannose; O-linked GalNAc; Core mannose and core fucose; Tri-antennary (2-4), Bi-antennary, Bisecting; Bi-antennary, Core mannose and core fucose; N-linked terminal GlcNAc, Sialic acid; High antennarity; Fucose (Lewis A, Lewis X and Lewis Y); and 2,3 sialic acid.

4. (canceled)

5. (canceled)

6. A method for detecting gastrointestinal cancer in a sample taken from a subject, the method comprising detecting a glycan in the sample taken from the subject, the glycan comprising a motif selected from the group consisting of Fucose, Sialyl Lewis A; High mannose, Bi-antennary; Tri/tetra-antennary; High mannose; O-linked GalNAc; Core mannose and core fucose; Tri-antennary (2-4), Bi-antennary, Bisecting; Bi-antennary, Core mannose and core fucose; N-linked terminal GlcNAc, Sialic acid; High antennarity; Fucose (Lewis A, Lewis X and Lewis Y); and 2,3 sialic acid.

7. The method of claim 6, wherein said glycan is characterized by having reactivity to a saccharide binding agent selected from the group consisting of: ALAA, AOL, Anti-sLeA, CONA, DC-SIGN, DSA, ECL, HHA, HPA, LCA, PHAE, PHAL, PSA, PVL, STL, Siglec-5, Siglec-7, UEAI and WGA.

8. (canceled)

9. A biomarker for detecting pancreatic cancer in a sample taken from a subject, comprising reactivity to a glycan on haptoglobin, wherein said reactivity relates to binding of one or more of HPA, bi1, LCA, WFA, gal-galnac2, Siglec-7 or comprising one or more glycans having reactivity to one or more of the following saccharide binding agent combinations: PSA and core 22; PHAL and core11; WGA and bi3; PHAL and bi2; PSA and bi2; PHAL and core1; PHAE and PHAL; or a combination or a ratio thereof.

10. The biomarker of claim 9, wherein the biomarkers are selected from the following analytical biomarker functions: Model 1-Log 2 PSA/Log 2 core22; Log 2 PHAL/Log 2 core11; Log 2 WGA/Log 2 bi3; log 2(PHAL/bi2). Model 2-Log 2 PSA/Log 2 bi2; Log 2 WGA/Log 2 bi3; Log 2 PHAL/Log 2 core1; log 2(PHAE/PHAL).

11. (canceled)

12. The biomarker of claim 9, comprising reactivity to a combination of one or more of HPA and bi1; LCA and HPA; WFA and gal-galnac2; or WFA and Siglec-7.

13. The biomarker of claim 12, wherein the biomarkers are selected from the following analytical biomarker functions: Model 1: log 2(HPA/bi1); log 2(LCA/HPA); log 2(WFA/gal_galnac2); and Model 2: log 2(WFA/gal_galnac2); log 2(WFA/Siglec-7); log 2(LCA/HPA).

14. (canceled)

15. The method of claim 6, further comprising contacting the sample with a saccharide binding agent according to any of the above claims; and if binding is detected, diagnosing the subject with cancer.

16. The method of claim 15, for early diagnosis and/or monitoring.

17. The method of claim 16, wherein said contacting the sample comprises applying the sample to a microarray; and detecting binding of a glycan in the sample to a lectin or antibody on said microarray.

18. The method of claim 17, wherein said microarray is printed on slides selected from the group consisting of nitrocellulose coated slides, epoxy slides or hydrogel coated slides.

19. The method of claim 17, wherein said gastrointestinal tract cancer comprises stomach cancer or pancreatic cancer.

20. The method of claim 6, wherein said sample is selected from the group consisting of seminal plasma, blood, serum, urine, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, cyst fluid, broncho alveolar lavage, lavage of the reproductive system and/or lavage of any other part of the body or system in the body, and stool or a tissue sample.

21. The method of claim 20, wherein said saccharide binding agent is an essentially sequence-specific agent.
